(19) United States
(12) Patent Application Publication
(10) Pub. No.: US 2007/0110751 A1
(43) Pub. Date: May 17, 2007

(54) Compositions and methods for reducing infarct size

(76) Inventors: Robb MacLellan, Sherman Oaks, CA (US); Peipei Ping, Marina del Rey, CA (US); David Liem, Los Angeles, CA (US); Samson Chan, Alhambra, CA (US)

Correspondence Address: NEEDLE & ROSENBERG, P.C., SUITE 1000, 999 PEACHTREE STREET, ATLANTA, GA, 30309-3915, US

(21) Appl. No.: 11/586,326

(22) Filed: Oct. 25, 2006

Related U.S. Application Data
(60) Provisional application No. 60/730,188, filed on Oct. 25, 2005.

(51) Int. Cl. A61K 39/395; A61K 31/55; A61K 31/522; A61K 31/353; A61K 31/513

(52) U.S. Cl. 424/146.1; 424/718; 514/44; 514/211.09; 514/215; 514/263.32; 514/269; 514/456

(57) ABSTRACT

Disclosed are compositions and methods for Disclosed are methods related to the use of Cdk2 inhibitors to reducing myocardial ischemic/reperfusion injury including but not limited to reduced infarct size. It is also disclosed that the same methods are equally appropriate for use in reducing injury following stroke including but not limited to (ischemic strokes (including strokes resulting from cerebral thrombosis, cerebral embolism, and atrial fibrillation), hemorrhagic strokes (including strokes resulting from aneurysm and arteriovenous malformation), and transient ischemic attack), reducing infarct size following pulmonary infarction, reducing renal ischemia injury, reducing ischemic/reperfusion injury occurring during cardiac surgery where a heart lung machine is used such as Coronary artery bypassing, and reducing ischemic/reperfusion injury occurring during the preservation of organs for transplant.

This application claims the benefit of U.S. Provisional Application Ser. No. 60/730,188, filed on Oct. 25, 2005, which is incorporated by reference herein in its entirety.

This work was supported by United States NIH grants AHA EIA 0340087N, R01 HL62448, R01 HL65431, and P01 HL080111. The government of the United States has certain rights in the invention.

### I. SUMMARY

Ischemia/reperfusion (I/R) injury to the heart is accompanied by the upregulation and posttranslational modification of a number of proteins normally involved in regulating cell cycle progression. Two such proteins, cyclin-dependent kinase-2 (Cdk2) and its downstream target, the retinoblastoma gene product (Rb), also play a critical role in the control of apoptosis. Myocardium ischemia activates Cdk2, resulting in the phosphorylation and inactivation of Rb. Disclosed herein, cell cycle proteins, Cdk2 signaling pathways, are critical regulators of cardiac I/R injury in vivo and support a cardioprotective role for Rb. Disclosed are methods related to the use of Cdk2 inhibitors to reducing myocardial ischemic/reperfusion injury including but not limited to reduced infarct size. It is also disclosed that the same methods are equally appropriate for use in reducing injury following stroke including but not limited to (ischemic strokes (including strokes resulting from cerebral thrombosis, cerebral embolism, and atrial fibrillation), hemorrhagic strokes (including strokes resulting from aneurysm and arteriovenous malformation), and transient ischemic attack), reducing infarct size following pulmonary infarction, reducing renal ischemia injury, reducing ischemic/reperfusion injury occurring during cardiac surgery where a heart lung machine is used such as Coronary artery bypassing, and reducing ischemic/reperfusion injury occurring during the preservation of organs for transplant.

### II. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows the activation of cell cycle-related proteins in ischemia-reperfusion injury. FIG. 1A shows that western blots were performed on protein lysates extracted from ventricular tissue harvested at the indicated time points. FIG. 1B shows that Cdk2-associated and kinase activities were analyzed by immune complex kinase assays using histones H1 as substrate. Complexes were immunoprecipitated from 300 μg of total protein lysates from each of the indicated treatment groups. ³²P-labeling of histones H1 was measured using a standard Cdk2 kinase assay. FIG. 1C shows that wildtype C57/BL6 mice were preconditioned by treating with a NO donor (DETA) or vehicle, 24 hours before I/R injury. Cdk2 kinase assays demonstrate reduced ³²P-labeling of purified histones consistent with decreased Cdk2 activity in DETA/NO-treated hearts after ischemia when compared to vehicle-treated hearts. IFS in DETA/NO-treated mice compared to control Vehicle-treated mice are shown (*P<0.05; n=4 per group).

FIG. 2 shows that Cdk2 activity induces apoptosis in NRVMs. FIG. 2A shows that neonatal cardiac myocytes were isolated and infected with the indicated virus and cultured in serum free media for 48 hours. Protein lysates were prepared, and Western or Cdk assays performed. FIG. 2B shows that DNA was isolated from NRVMs infected with the indicated virus and separated on a 1.2% agarose gel. FIG. 2C shows that Caspase-3 activity was determined using a colorimetric assay on lysates prepared from infected NRVMs. Caspase-3 activity in AdCycA/Cdk2 infected NRVMs was increased when compared to uninfected control or AdLacZ infected NRVMs (*P<0.001, n=3 reps).

FIG. 3 shows that inhibition of Cdk2 attenuates infarct size in vivo. FIG. 3A shows that C57/BL6 mice were subjected to 30-min coronary occlusion followed by 24 h of reperfusion. Mice were injected intraperitoneally two hours prior to ischemia with 2.8 mg/gm of Roscovitine or DMSO carrier. Cdk2 kinase assays demonstrate reduced ³²P-labeling of purified histones consistent with decreased Cdk2 activity in Roscovitine-treated hearts after I/R when compared to vehicle-treated hearts. IFS in Roscovitine-treated mice compared to control vehicle-treated mice are shown (*P<0.001; n=6 per group). FIG. 3B shows that wildtype or Cdk2-null mice were subjected to I/R injury. Ventricular lysates were probed for the indicated proteins. Mean IFS sizes after 24 hours of reperfusion are shown. (*P<0.05 for Cdk2^{+/+} versus Cdk2^{-/-} IFS; n=5 per group).

FIG. 4 shows that apoptosis is reduced in ischemic Cdk2^{-/-} myocardium. FIG. 4A shows immunofluorescent staining for TUNEL (green) and cardiac-specific marker MF20 (red) was performed on myocardial sections from wildtype or Cdk2-null mice subjected to I/R. FIG. 4B shows the percentage of TUNEL positive nuclei was quantified on myocardial sections from the indicated genotypes and treatments. The results from examination of at least 3,500 nuclei per animal are shown. (*P<0.05 Cdk2^{+/+} after I/R versus Cdk2^{+/+} or Cdk2^{-/-} at baseline and P=0.0.5 for Cdk2^{+/+} versus Cdk2^{-/-} after I/R; n=4 per group).

FIG. 5 shows that ischemic injury is enhanced in Rb-null myocardium. FIG. 5A shows representative TTC-stained hearts from control (CRb^{+/+}) or cardiac-restricted Rb-deficient (CRb^{L/L}) mice after I/R injury are shown. FIG. 5B shows that CRb^{+/+} or CRbL^{L/L} mice were subjected to ischemia reperfusion injury. Mean IFS sizes after 24 hours of reperfusion are shown. (*P<0.001 for CRb^{L/L} versus CRb^{+/+} IFS; n=8 per group). FIG. 5C shows that the frequency of TUNEL positive myocyte nuclei for each animal was determined by examining at least 7,500 myocardial nuclei. Caspase 3 activity was determined on ventricular lysates from ischemic CRb^{+/+} or CRb^{L/L} mice and the results shown. (*P<0.05 for CRb^{L/L} versus CRb^{+/+}, n=4 per group).

FIG. 6 shows that caspase-resistant Rb mutant mice are indistinguishable from control mice. FIG. 6A shows schematic diagram illustrating the point mutation created to induce caspase resistance and results of PCR genotyping. FIG. 6B shows representative TTC-stained hearts from wildtype (Rb^{+/+}) or caspase-resistant Rb mutant (Rb^{MI/MI}) mice after I/R injury are shown. FIG. 7C shows that Rb^{+/+} or Rb^{MI/MI} mice were subjected to ischemia reperfusion injury. Mean IFS sizes after 24 hours of reperfusion are shown. (P=n.s.; n=5 per group).

FIG. 7 shows altered expression of apoptotic regulatory proteins in ischemic Rb-deficient hearts. FIGS. 7A shows representative Westerns performed on ventricular lysates from the indicated mice. FIG. 7B shows hearts from each genotype and condition were probed and the results were quantified using enhanced chemiluminescence. (*P<0.05 for ischemic CRb^{+/+} versus nonischemic CRb^{+/+} ventricles, **P<0.05 for ischemic CRb^{L/L} versus nonischemic CRb^{L/L} or ischemic CRb^{+/+} ventricles; n=4 per group).

### III. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

B. Methods of Reducing Cardiac Ischemia/Reperfusion Injury

Herein disclosed are methods of reducing ischemia/reperfusion injury in a subject in need thereof comprising administering to the subject an agent that inhibits cyclin dependent kinase 2 (Cdk2) activity. It is understood and herein contemplated that ischemia is a deficiency of blood in a part, usually due to functional constriction or actual obstruction of a blood vessel. Such a deficiency result in an infarct, an area of cell death in a tissue due to local ischemia resulting from obstruction of circulation to the area, most commonly by a thrombus or embolus. When the constriction or obstruction is removed, and blood flow restored reperfusion has occurred. Although blood flow is restored, the reperfusion can also result in adverse effects of the restoration of blood flow following an ischemic episode, including cellular swelling and necrosis, apoptosis, edema, hemorrhage, the no-reflow phenomenon, and tissue damage by free oxygen radicals. Thus, one manifestation of reducing ischemia/reperfusion injury is reducing infarct size. Therefore, disclosed herein are methods of reducing infarct size following an ischemia/reperfusion event in a subject comprising administering to the subject an agent that inhibits Cdk2 activity.

It is understood that there are many known causes of ischemia/reperfusion injury. For example, an ischemic/reperfusion injury can result from ischemia reperfusion event such as myocardial inschemia, myocardial reperfusion, subendocardial ischemia, Takayasu's arteritis, including but not limited to (ischemic strokes (including strokes resulting from cerebral thrombosis, cerebral embolism, and atrial fibrillation), hemorrhagic strokes (including strokes resulting from aneurysm and arteriovenous malformation), and transient ischemic attack), pulmonary infarction, hypoxia, retinal ischemia, renal ischemia, cardiac surgery where a heart lung machine is used such as Coronary artery bypassing, and preservation of organs for transplant. Thus, also disclosed herein are methods of reducing ischemia/reperfusion injury comprising administering an agent that inhibits Cdk2 activity, wherein the ischemia/reperfusion injury occurs following an ischemia/reperfusion event selected from the group consisting of myocardial inschemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypozia, retinal ischemia, renal ischemia, ischemic/reperfusion event occurring during cardiac surgery where a heart lung machine is used such as Coronary artery bypassing, and ischemic/reperfusion events occurring during the preservation of organs for transplant.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. It is understood and herein contemplated that “treatment” does not necessarily refer to a cure of the disease or condition nor a complete prevention of infarct, but can involve, for example, an improvement in the outlook of an ischemia/reperfusion injury. The effects of treatment can be measured or assessed as described herein and as known in the art as is suitable for the disease, pathological condition, or disorder involved. Such measurements and assessments can be made in qualitative and/or quantitiative terms. Thus, for example, characteristics or features of a disease, pathological condition, or disorder and/or symptoms of a disease, pathological condition, or disorder can be reduced to any effect or to any amount.

Also, for example, treating ischemia/reperfusion injury can comprise any method or the administration of any agent that affects tissue damage resulting from apoptosis triggered by Cdk2 activation in a manner that ameliorates the degree of or potential for tissue injury associated with an ischemia/reperfusion event. For example, administration of Roscovitine to inhibit Cdk2 activity.

The term “decrease” in the context of Cdk2 can refer to any change that results in a smaller amount of Cdk2 activity. Thus, a “decrease” can refer to a reduction in an activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed.

“Reducing,” “reduce,” or “reduction” in the context of a disease or condition herein refers to a decrease in the cause, symptoms, or effects of a disease or condition. Therefore, in the disclosed methods, “reducing” can refer to a 10%, 20%, 30%, 40%, 50%, 60%, 70%, 80%, 90%, or 100% decrease in the amount of injury due to ischemia/reperfusion including but not limited to infarct size.

Unfortunately, ischemia/reperfusion events can occur in subjects who are unaware of the impending infarction. Often the first indication to such individuals is the ischemia/reperfusion event itself. In such individuals, there is a need to reduce the potential ischemia/reperfusion injury. Thus, it is herein contemplated that disclosed methods can be used to reduce ischemia/reperfusion injury following the ischemia/reperfusion event. Thus, for example, disclosed are methos of reducing ischemia/reperfusion injury in a subject in need thereof comprising administering to the subject an agent that inhibits Cdk2 activity, wherein the agent is administered within 24 hours following the ischemia/reperfusion event. It is understood that the more quickly the agent can be administered following the ischemia/reperfusion event, the less the likelihood of injury and subsequently the greater the potential reduction in infarct size. Thus, disclosed herein are methods wherein the agent is administered within 24, 12, 6, 2, 1 hour(s), 30, 15, 10, 5 minutes following the ischemia/reperfusion event. It is understood that administration of the agent can occur at any time between 5 minutes and 24 hours following the ischemia/reperfusion event. It is also understood that ischemia and reperfusion are not only physiologically different events, but do not necessarily occur at the same time. As ischemia refers to deficiency of blood to a part typically due to a thrombus or emobolus and reperfusion injury results when the obstruction or constriction is removed, it is possible and desireable to reduce ischemia/reperfusion injury during the ischemia/reperfusion event. Thus, for example, a Cdk2 inhibitor could be administered during the ischemia or alternatively after the ischemia, but before reperfusion has occurred, or alternatively after the ischemia and at the time of reperfusion. Thus, disclosed herein are methods wherein the agent is administered during the ischemia/reperfusion event.

It is understood and herein contemplated that typically the greatest reduction in ischemia/reperfusion injury will occur in individuals where the Cdk2 inhibitor is administered before the ischemia/reperfusion event. Thus, it is understood and contemplated herein that individuals at risk for or having a history of ischemia/reperfusion events can decrease the risk of further necrosis in future events by taking the Cdk2 inhibitor prophylactically. It is also understood that many ischemia/reperfusion events have early warning symptoms preceeding the actual event which when recognized can allow the subject to seek immediate treatment. Even if there is ischemic/reperfusion injury caused by future ischemia/reperfusion events, it is contemplated that the prophylactic administration of Cdk2 inhibitors will reduce infarct size. For example, disclosed herein are methods of reducing ischemia/reperfusion injury in a subject in need thereof comprising administering to the subject an agent that inhbits Cdk2 activity, wherein the agent is administered at least 30 minutes before the ischemia/reperfusion event. Thus, disclosed herein are methods wherein the agent is administered 15, 30 minutes, 1, 2, 6, 12, 24 hour(s), 2, 3 days, 1, or 2 weeks or any time point in between before the ischemia/reperfusion event.

1. CDK2

As set forth in U.S. Pat. No. 6,486,166, which is incorporated herein by reference in its entirety, cyclin dependent kinases (Cdks) comprise a family of at least 10-12 enzymes. Cdk2, Cdk4, and Cdk6 are thought to play critical roles in G1 phase progression. It is generally believed that Cdk4 and Cdk6 regulate processes that are essential for progression through mid to late G1 phase; whereas Cdk2 regulates processes that are involved in the initiation of S phase. Cdk4 and Cdk6 are activated by association with one or another of the D-type (D1, D2, D3) cyclins. Cdk2 is activated primarily by association with cyclin E or cyclin A.

Cyclin binding to Cdks is a prerequisite for covalent modifications that are essential for catalytic activity. For example, binding of an appropriate cyclin is required in order for Cdk-activating kinase (CAK) to phosphorylate T160 on Cdk2 and T174 on Cdk4. The Cdks are inactive unless these carboxy-terminal threonine residues are phosphorylated. Cyclin kinase inhibitors act in part to block CAK-dependent activation of Cdks, suggesting that cyclins and cyclin kinase inhibitors serve antagonistic functions with respect to CAK-dependent activation of Cdks.

2. CDK2 Inhibitors

Herein, “inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

It is understood and herein contemplated that one manner by which Cdk2 activity can be inhibited is through the use of a Cdk2 inhbitor. Such molecules are known to the skilled artisan and include agents such as purine analogs or derivatives thereof, pyrimidine analogs or derivatives thereof, flavones, oxindoles, starurosporine, diarylureas, and paullones. Substituted adenines such as Roscovitine (6-Benzylamino-2-(R)-[(1-ethyl)-2-hydroxyethylamino]-9-isopropyl-purine), Olomoucine (6-(Benzylamino)-2-(2-hydroxyethylamino)-9-methylpurine), N⁹-Isopropylolomoucine (2-(2′-Hydroxyethylamino)-6-benzylamino-9-isopropylpurine), and N6-Isopentyladenine; Phenylaminopyrimidines such as CGP60474 ( ) and CINK4 ( ); thiazolopyrimidines; Purvalanols which contain 6-anilino rather than 6-benzylamino purine substituents such as Purvalanol A ((2R)-2-[[6-[(3-Chlorophenyl)amino]-9-(1-methylethyl)-9H-purin-2-yl]amino]-3-methyl-1-butanol) and Purvalanol B ( ); Guanine derivatives including NU20580 ( ) and NU6102 (O6-Cyclohexylmethyl-2-(4′-sulfamoylanilino)purine); Nitrosopyrimidine NU6027( ); purine-based CDK inhibitors, such as CGP79807 and CGP74514 (N²-(cis-2-Aminocyclohexyl)-N⁶-(3-chlorophenyl)-9-ethyl-9H-purine-2,6-diamine hydrochloride); Aloisine A (7-n-Butyl-6-(4-hydroxyphenyl)[5H]pyrrolo[2,3-b]pyrazine); compounds containing 7,12-dihydro-indolo[3,2-d][1]benzazepin-6(5H)-one also known as paullones such as Kenpaullone (9-Bromo-7,12-dihydroindolo[3,2-d][1]benzazepin-6(5H)-one) and alsterpaullone (9-Nitro-7,12-dihydroindolo-[3,2-d][1]benzazepin-6(5)-one); indirubin-3′-monoxime; oxindole-based CDK inhibitors such as phenylhydrazone, anilinomethylene oxindoles, and indirubin 5-sulfonate; fused thiazole derivative GW8510 (4-{[(7-Oxo-6,7-dihydro-8H-[1,3]thiazolo[5,4-e]indol-8-ylidene)methyl]amino}-N-(2-pyridyl)benzenesulfonamide); indenopyrazoles; flavonoids such as flavopiridol and thio-and oxo-containing analogs; starurosporine; butyrolactone-I; and diarylureas.

Additional illustrative cyclin-dependent kinase 2 inhibitors that may be employed in the broad practice of the invention to reduce myocardial ischemic/reperfusion injury, reduce infarct size following myocardial ischemia/reperfusion, subendocardial ischemia, Takayasu's arteritis, reduce injury following stroke including but not limited to (ischemic strokes (including strokes resulting from cerebral thrombosis, cerebral embolism, and atrial fibrillation), hemorrhagic strokes (including strokes resulting from aneurysm and arteriovenous malformation), and transient ischemic attack), reduce infarct size following hypoxia, reduce infarct size following pulmonary infarction, reduce renal ischemia injury, reduce ischemic/reperfusion injury occurring during cardiac surgery where a heart lung machine is used such as Coronary artery bypassing, and reduce ischemic/reperfusion injury occurring during the preservation of organs for transplant, include the cyclin-dependent kinase 2 inhibitor compounds described in the following references, as well as in the references identified in the bibliography set forth hereinafter, the disclosures of all of which are hereby incorporated herein by reference in their respective entireties: U.S. Pat. No. 7,105,529 for its teachings of Cdk2 inhibitors, their structure, administration, and analogs thereof; substituted oxindole derivatives described in International Patent Application No. PCT/EP98/05559; purine derivatives described in International Publication WO97/20842 of CNRS Center Natural Research; pyridylpyrimidinamine derivatives described in International Publication WO95/09852; 2,6,9-trisubstituted compounds described in International Publication WO98/05335; 4H-1-benzopyran-4-one derivatives described in German Patent 3836676; 2-thiol and 2-oxo-flavopiridol analogues described in U.S. Pat. No. 5,705,350, and in U.S. Pat. No. 5,849,733; pyrido [2,3-D] pyrimidines and 4-aminopyrimidines described in International Publication WO98/33798 as well as in U.S. Pat. Nos. 5,776,942; 5,733,913; 5,223,503; 4,628,089; 4,536,575; 4,431,805; and 4,252,946; antiviral CDK2 inhibitor compounds described in International Publication WO98/39007; chimeric CDK2 inhibitors described in International Publication WO97/27297; the peptide inhibitors described in U.S. Pat. No. 5,625,031; and CDK2 inhibitor antisense sequences described in U.S. Pat. No. 5,821,234.

It is also understood that inhibitors of Cdk2 can include metazoan cyclin kinase inhibitors (CKIs). Broadly speaking, there are two classes of CKIs. One class consists of the members of the INK family, which include but is not limited to p15, p16, p18, and p19. INK type CKIs bind to and sequester Cdk4 and Cdk6, but not Cdk2. The sequestration of the Cdk subunit by these inhibitors prevents Cdk4 or 6 from complexing with its cyclin subunit. The INK type inhibitors also prevent Cdk4 and Cdk6 phosphorylation by dissociating the cyclin/Cdk binary complex which is the substrate for CAK. Activated cyclinD/Cdk4 complexes (i.e., those in which T174 has already been phosphorylated) are also inhibited due to displacement of the cyclin subunit by these inhibitors.

The second family of CKIs, is comprised of Cip1 (also called WAF1, Cap20, and Sdi1) Kip1 and Kip2. Examples of such inhibitors include p21cip1/WAF, p27kip1, and p57kip2. These CKIs bind to cyclin/Cdk binary complexes that contain Cdk2, Cdk3, Cdk4, or Cdk6. Unlike members of the INK family, Cip1 and Kip1 do not disrupt cyclin/Cdk complexes, but bind to such entities to form ternary and higher order complexes. Cip1 and Kip1 are structurally related to each other, but not to the INK-type CKIs. Both Cip1 and Kip1 have similar properties in vitro. Both inhibit phosphorylation by CAK at low stoichiometries, thereby preventing Cdk activation. Cip1 appears to facilitate the formation of cyclin A/Cdk2 complexes at low concentrations of the inhibitor, and it has been suggested that Cip1 may be involved in recruitment of cyclin A and Cdk2 to newly synthesized ternary complexes. At higher stoichiometries. Cip1 and Kip1 are potent inhibitors of activated Cdk2, Cdk3, Cdk4. and Cdk6.

Thus, disclosed herein are methods wherein the agent is selected from the group consisting of purine analogs or derivatives thereof, pyrimidine analogs or derivatives thereof, flavones, oxindoles, starurosporine, diarylureas, and paullones. Additionally, it is understood that other inhibitors such as anti-cdk2 antibodies, RNA mimetics, and nitric oxide can be used to inhibit Cdk2 activity in the disclosed methods.

3. Retinoblastoma

The mechanism whereby Cdks regulate cell cycle progression centers around the product of the retinoblastoma tumor suppressor gene, Rb. Rb functions in part by serving as a control point that connects extracellular signals and gene transcription. Rb is a phosphoprotein that is differentially phosphorylated throughout the cell cycle. During G0 or early G1, Rb is present in a hypophosphorylated form and exerts its growth suppressive activity. When Rb is present in this form it binds to and inactivates certain members of the E2F transcription factor family. The binding of Rb with E2F blocks E2F-mediated transcription of cellular genes that are required for entry into S phase, such as DNA polymerase ax, and dihydrofolate reductase. G1 cyclin/Cdk complexes are believed to relieve this Rb-mediated suppression by phosphorylating Rb, which results in the release of E2F. Upon its release E2F can then activate transcription of cellular genes essential for entry into S phase. Thus, disclosed herein are methods of reducing ischemia/reperfusion injury in a subject in need thereof comprising administering to the subject an agent that inhibits phosphorylation of retinoblastoma protein (Rb). Also disclosed are methods of reducing infarct size following an ischemia/reperfusion event in a subject comprising administering to the subject an agent that inhibits phosphorylation of Rb.

It is understood that there are many known causes of ischemia/reperfusion injury. For example, an ischemic/reperfusion injury can result from ischemia reperfusion event such as myocardial inschemia, myocardial reperfusion, subendocardial ischemia, Takayasu's arteritis, including but not limited to (ischemic strokes (including strokes resulting from cerebral thrombosis, cerebral embolism, and atrial fibrillation), hemorrhagic strokes (including strokes resulting from aneurysm and arteriovenous malformation), and transient ischemic attack), pulmonary infarction, hypoxia, retinal ischemia, renal ischemia, ischemia/reperfusion events occurring during cardiac surgery where a heart lung machine is used such as Coronary artery bypassing, and ischemia/reperfusion events occurring during the preservation of organs for transplant. Thus, also disclosed herein are methods of reducing ischemia/reperfusion injury comprising administering an agent that phosphorylation of Rb, wherein the ischemia/reperfusion injury occurs following an ischemia/reperfusion event selected from the group consisting of myocardial inschemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypozia, retinal ischemia, renal ischemia, ischemia/reperfusion events occurring during cardiac surgery where a heart lung machine is used such as Coronary artery bypassing, and ischemia/reperfusion events occurring during the preservation of organs for transplant.

As previously disclosed above, it is herein contemplated that disclosed methods can be used to reduce ischemia/reperfusion injury following the ischemia/reperfusion event. Thus, for example, disclosed are methos of reducing ischemia/reperfusion injury in a subject in need thereof comprising administering to the subject an agent that inhibits phosphorylation of Rb, wherein the agent is administered within 24 hours following the ischemia/reperfusion event. It is understood that the more quickly the agent can be administered following the ischemia/reperfusion event, the less the likelihood of injury and subsequently the greater the potential reduction in infarct size. Thus, disclosed herein are methods wherein the agent is administered within 24, 12, 6, 2, 1 hour(s), 30, 15, 10, 5 minutes following the ischemia/reperfusion event. It is understood that administration of the agent can occur at any time between 5 minutes and 24 hours following the ischemia/reperfusion event. It is also understood that ischemia and reperfusion are not only physiologically different events, but do not necessarily occur at the same time. As ischemia refers to deficiency of blood to a part typically due to a thrombus or emobolus and reperfusion injury results when the obstruction or constriction is removed, it is possible and desireable to reduce ischemia/reperfusion injury during the ischemia/reperfusion event. Thus, for example, a Rb inhibitor could be administered during the ischemia or alternatively after the ischemia, but before reperfusion has occurred, or alternatively after the ischemia and at the time of reperfusion. Thus, disclosed herein are methods wherein the agent is administered during the ischemia/reperfusion event.

As disclosed above, it is understood and herein contemplated that typically the greatest reduction in ischemia/reperfusion injury will occur in individuals where the Rb inhibitor is administered before the ischemia/reperfusion event. Thus, it is understood and contemplated herein that individuals at risk for or having a history of ischemia/repersution events can decrease the risk of further necrosis in future events by taking the inhibitor Rb phosphorylation prophylactically. For example, disclosed herein are methods of reducing ischemia/reperfusion injury in a subject in need thereof comprising administering to the subject an agent that inhbits phosphorylation of Rb, wherein the agent is administered at least 30 minutes before the ischemia/reperfusion event. Thus, disclosed herein are methods wherein the agent is administered 15, 30 minutes, 1, 2, 6, 12, 24 hour(s), 2, 3 days, 1, or 2 weeks or any time point in between before the ischemia/reperfusion event.

It is understood that inhibitors of the phosphorylation of Rb can take many forms such as an antibody that blocks the phosphorylation sites used by Cdk2 to inactivate Rb or an RNA mimetic that mimics the phosphorylation site. Thus disclosed herein are methods of reducing ischemia/reperfusion injury in a subject in need thereof comprising administering to the subject an agent that inhibits phosphorylation of retinoblastoma protein (Rb), wherein the agent is an anti-Rb antibody or RNA mimetic.

C. Methods of Screening

It is understood and herein contemplated that the disclosed effects of Cdk2 and Rb activity can be used to screen for agents that can reduce ischemia/reperfusion injury. Thus, disclosed herein are methods of screening for an agent that reduces ischemia/reperfusion injury comprising administering an agent to a subject, inducing ischemia/reperfusion, and measuring the activity of Cdk2, wherein a decrease in the Cdk2 activity relative to a control indicates an agent that reduces ischemia/reperfusion injury. It is also understood that one type of ischemia/reperfusion injury is infarction. Thus, also disclosed are methods of screening for an agent that reduces infarct size following ischemia/reperfusion comprising administering an agent to a subject, inducing ischemia/reperfusion, and measuring the activity of Cdk2, wherein a decrease in the Cdk2 activity relative to a control indicates an agent that can reduce infarct size. It is understood that ischemia/reperfusion injury can be induced by any means known to those of skill in the art such as ligation (for example, coronary ligation). It is understood that the subject can be any mammal including but not limited to mouse, rat, rabbit, guinea pig, cow, horse, pig, cat, dog, monkey, chimpanzee, or human. It is also understood that Cdk2 activity can be assessed by any method known now by the skilled artisan or developed in the future to assess protein activity such as Western Blot or Cdk kinase assay.

D. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Cdk2 inhibitor is disclosed and discussed and a number of modifications that can be made to a number of molecules including the Cdk2 inhibitor are discussed, specifically contemplated is each and every combination and permutation of Cdk2 inhibitor and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

1. Homology/identity

It is understood that one way to define any known variants and derivatives or those that might arise, of the disclosed genes and proteins herein is through defining the variants and derivatives in terms of homology to specific known sequences. For example SEQ ID NO. 1 sets forth a particular sequence of a Cdk2 and SEQ ID NO. 2 sets forth a particular sequence of the protein encoded by SEQ ID NO. 1, a Cdk2 protein. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

2. Nucleic Acids

There are a variety of molecules disclosed herein that are nucleic acid based, including for example the nucleic acids that encode, for example, Cdk2 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an intemucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, Cdk2 or Retinoblastoma protein as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA, genomic DNA, or the polypeptide comprising Cdk2 as exemplified by SEQ ID Nos 1 and 2. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d})less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos.: 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{ds} from the target molecule of less than 10⁻¹² M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. Representative examples of how to make and use aptamers to bind a variety of different target molecules can be found in the following non-limiting list of 30 U.S. Pat. Nos.: 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos.: 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos.: 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos.: 5,595,873 and 5,652,107). There are also a number of ribozymes that are not found in natural systems, but which have been engineered to catalyze specific reactions de novo (for example, but not limited to the following U.S. Pat. Nos.: 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos.: 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰ or 10⁻¹². Representative examples of how to make and use triplex forming molecules to bind a variety of different target molecules can be found in the following non-limiting list of U.S. Pat. Nos.: 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukarotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules be found in the following non-limiting list of U.S. Pat. Nos.: 5,168,053, 5,624,824, 5,683,873, 5,728,521, 5,869,248, and 5,877,162.

3. Peptides

a) Protein Variants

As discussed herein there are numerous variants of the Cdk2 protein and Rb protein that are known and herein contemplated. In addition, to the known functional Cdk2 strain variants there are derivatives of the Cdk2 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations generally should not place the sequence out of reading frame (unless a truncated protein is desired) and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic | Glu |
| acidp | |
| serine | SerS |
| threonine | ThrT |
| tyrosine | TyrY |
| tryptophan | TrpW |
| valine | ValV |

TABLE 2
Amino Acid Substitutions
Original Residue Exemplary Conservative Substitutions,
others are known in the art.

Alaser
Arglys, gln
Asngln; his
Aspglu
Cysser
Glnasn, lys
Gluasp
Glypro
Hisasn; gln
Ileleu; val
Leuile; val
Lysarg; gln;
MetLeu; ile
Phemet; leu; tyr
Serthr
Thrser
Trptyr
Tyrtrp; phe
Valile; leu

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO: 1 sets forth a particular sequence of Cdk2 and SEQ ID NO: 2 sets forth a particular sequence of a Cdk2 protein. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. For example, one of the many nucleic acid sequences that can encode the protein sequence set forth in SEQ ID NO: 2 is set forth in SEQ ID NO: 1. It is also understood that while no amino acid sequence indicates what particular DNA sequence encodes that protein within an organism, where particular variants of a disclosed protein are disclosed herein, the known nucleic acid sequence that encodes that protein is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Biotechnology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂ —, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH2—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

4. Antibodies 1

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with Cdk2 or Rb such that Cdk2 is inhibited from interacting with Rb. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

(2) Human Antibodies

The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

(3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al., Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

(4) Administration of Antibodies

Administration of the antibodies can be done as disclosed herein. Nucleic acid approaches for antibody delivery also exist. The broadly neutralizing anti-Cdk2 antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

5. Pharmaceutical Carriers/Delivery of Pharamceutical Products

As described herein, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms/disorder are/is effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed composition, such as an antibody, for treating, inhibiting, or preventing an ischemia/reperfusion injury, the efficacy of the therapeutic antibody can be assessed in various ways well known to the skilled practitioner. For instance, one of ordinary skill in the art will understand that a composition, such as an antibody, disclosed herein is efficacious in inhibiting Cdk2 activity or inhibiting Rb phosphorylation in a subject by observing that the composition reduces infarct size.

6. Compositions Identified by Screening with Disclosed Compositions/Combinatorial Chemistry

a) Combinatorial Chemistry

The disclosed Cdk2 and Rb proteins can be used as targets for any combinatorial technique to identify molecules or macromolecular molecules that interact with the disclosed compositions in a desired way. The nucleic acids, peptides, and related molecules disclosed herein can be used as targets for the combinatorial approaches. Also disclosed are the compositions that are identified through combinatorial techniques or screening techniques in which the compositions disclosed in SEQ ID NOS:1-4 or portions thereof, are used as the target in a combinatorial or screening protocol.

It is understood that when using the disclosed compositions in combinatorial techniques or screening methods, molecules, such as macromolecular molecules, will be identified that have particular desired properties such as inhibition of Cdk2 activity or inhibition of Rb phosphorylation. The molecules identified and isolated when using the disclosed compositions are also disclosed. Thus, the products produced using the combinatorial or screening approaches that involve the disclosed compositions, such as, Cdk2 and Rb, are also considered herein disclosed.

It is understood that the disclosed methods for identifying molecules that inhibit the interactions between, for example, Cdk2 and Rb can be performed using high through put means. For example, putative inhibitors can be identified using Fluorescence Resonance Energy Transfer (FRET) to quickly identify interactions. The underlying theory of the techniques is that when two molecules are close in space, i.e., interacting at a level beyond background, a signal is produced or a signal can be quenched. Then, a variety of experiments can be performed, including, for example, adding in a putative inhibitor. If the inhibitor competes with the interaction between the two signaling molecules, the signals will be removed from each other in space, and this will cause a decrease or an increase in the signal, depending on the type of signal used. This decrease or increasing signal can be correlated to the presence or absence of the putative inhibitor. Any signaling means can be used. For example, disclosed are methods of identifying an inhibitor of the interaction between any two of the disclosed molecules comprising, contacting a first molecule and a second molecule together in the presence of a putative inhibitor, wherein the first molecule or second molecule comprises a fluorescence donor, wherein the first or second molecule, typically the molecule not comprising the donor, comprises a fluorescence acceptor; and measuring Fluorescence Resonance Energy Transfer (FRET), in the presence of the putative inhibitor and the in absence of the putative inhibitor, wherein a decrease in FRET in the presence of the putative inhibitor as compared to FRET measurement in its absence indicates the putative inhibitor inhibits binding between the two molecules. This type of method can be performed with a cell system as well.

Combinatorial chemistry includes but is not limited to all methods for isolating small molecules or macromolecules that are capable of binding either a small molecule or another macromolecule, typically in an iterative process. Proteins, oligonucleotides, and sugars are examples of macromolecules. For example, oligonucleotide molecules with a given function, catalytic or ligand-binding, can be isolated from a complex mixture of random oligonucleotides in what has been referred to as “in vitro genetics” (Szostak, TIBS 19:89, 1992). One synthesizes a large pool of molecules bearing random and defined sequences and subjects that complex mixture, for example, approximately 10¹⁵ individual sequences in 100 μg of a 100 nucleotide RNA, to some selection and enrichment process. Through repeated cycles of affinity chromatography and PCR amplification of the molecules bound to the ligand on the column, Ellington and Szostak (1990) estimated that 1 in 10¹⁰ RNA molecules folded in such a way as to bind a small molecule dyes. DNA molecules with such ligand-binding behavior have been isolated as well (Ellington and Szostak, 1992; Bock et al, 1992). Techniques aimed at similar goals exist for small organic molecules, proteins, antibodies and other macromolecules known to those of skill in the art. Screening sets of molecules for a desired activity whether based on small organic libraries, oligonucleotides, or antibodies is broadly referred to as combinatorial chemistry. Combinatorial techniques are particularly suited for defining binding interactions between molecules and for isolating molecules that have a specific binding activity, often called aptamers when the macromolecules are nucleic acids.

There are a number of methods for isolating proteins which either have de novo activity or a modified activity. For example, phage display libraries have been used to isolate numerous peptides that interact with a specific target. (See for example, U.S. Pat. Nos. 6,031,071; 5,824,520; 5,596,079; and 5,565,332 which are herein incorporated by reference at least for their material related to phage display and methods relate to combinatorial chemistry)

A preferred method for isolating proteins that have a given function is described by Roberts and Szostak (Roberts R. W. and Szostak J. W. Proc. Natl. Acad. Sci. USA, 94(23)12997-302 (1997). This combinatorial chemistry method couples the functional power of proteins and the genetic power of nucleic acids. An RNA molecule is generated in which a puromycin molecule is covalently attached to the 3′-end of the RNA molecule. An in vitro translation of this modified RNA molecule causes the correct protein, encoded by the RNA to be translated. In addition, because of the attachment of the puromycin, a peptdyl acceptor which cannot be extended, the growing peptide chain is attached to the puromycin which is attached to the RNA. Thus, the protein molecule is attached to the genetic material that encodes it. Normal in vitro selection procedures can now be done to isolate functional peptides. Once the selection procedure for peptide function is complete traditional nucleic acid manipulation procedures are performed to amplify the nucleic acid that codes for the selected functional peptides. After amplification of the genetic material, new RNA is transcribed with puromycin at the 3′-end, new peptide is translated and another functional round of selection is performed. Thus, protein selection can be performed in an iterative manner just like nucleic acid selection techniques. The peptide which is translated is controlled by the sequence of the RNA attached to the puromycin. This sequence can be anything from a random sequence engineered for optimum translation (i.e. no stop codons etc.) or it can be a degenerate sequence of a known RNA molecule to look for improved or altered function of a known peptide. The conditions for nucleic acid amplification and in vitro translation are well known to those of ordinary skill in the art and are preferably performed as in Roberts and Szostak (Roberts R. W. and Szostak J. W. Proc. Natl. Acad. Sci. USA, 94(23)12997-302 (1997)).

Another preferred method for combinatorial methods designed to isolate peptides is described in Cohen et al. (Cohen B. A., et al., Proc. Natl. Acad. Sci. USA 95(24):14272-7 (1998)). This method utilizes and modifies two-hybrid technology. Yeast two-hybrid systems are useful for the detection and analysis of protein:protein interactions. The two-hybrid system, initially described in the yeast Saccharomyces cerevisiae, is a powerful molecular genetic technique for identifying new regulatory molecules, specific to the protein of interest (Fields and Song, Nature 340:245-6 (1989)). Cohen et al., modified this technology so that novel interactions between synthetic or engineered peptide sequences could be identified which bind a molecule of choice. The benefit of this type of technology is that the selection is done in an intracellular environment. The method utilizes a library of peptide molecules that attached to an acidic activation domain.

Using methodology well known to those of skill in the art, in combination with various combinatorial libraries, one can isolate and characterize those small molecules or macromolecules, which bind to or interact with the desired target. The relative binding affinity of these compounds can be compared and optimum compounds identified using competitive binding studies, which are well known to those of skill in the art.

Techniques for making combinatorial libraries and screening combinatorial libraries to isolate molecules which bind a desired target are well known to those of skill in the art. Representative techniques and methods can be found in but are not limited to U.S. Pat. Nos. 5,084,824, 5,288,514, 5,449,754, 5,506,337, 5,539,083, 5,545,568, 5,556,762, 5,565,324, 5,565,332, 5,573,905, 5,618,825, 5,619,680, 5,627,210, 5,646,285, 5,663,046, 5,670,326, 5,677,195, 5,683,899, 5,688,696, 5,688,997, 5,698,685, 5,712,146, 5,721,099, 5,723,598, 5,741,713, 5,792,431, 5,807,683, 5,807,754, 5,821,130, 5,831,014, 5,834,195, 5,834,318, 5,834,588, 5,840,500, 5,847,150, 5,856,107, 5,856,496, 5,859,190, 5,864,010, 5,874,443, 5,877,214, 5,880,972, 5,886,126, 5,886,127, 5,891,737, 5,916,899, 5,919,955, 5,925,527, 5,939,268, 5,942,387, 5,945,070, 5,948,696, 5,958,702, 5,958,792, 5,962,337, 5,965,719, 5,972,719, 5,976,894, 5,980,704, 5,985,356, 5,999,086, 6,001,579, 6,004,617, 6,008,321, 6,017,768, 6,025,371, 6,030,917, 6,040,193, 6,045,671, 6,045,755, 6,060,596, and 6,061,636.

Combinatorial libraries can be made from a wide array of molecules using a number of different synthetic techniques. For example, libraries containing fused 2,4-pyrimidinediones (U.S. Pat. No. 6,025,371) dihydrobenzopyrans (U.S. Pat. Nos. 6,017,768 and 5,821,130), amide alcohols (U.S. Pat. No. 5,976,894), hydroxy-amino acid amides (U.S. Pat. No. 5,972,719) carbohydrates (U.S. Pat. No. 5,965,719), 1,4-benzodiazepin-2,5-diones (U.S. Pat. No. 5,962,337), cyclics (U.S. Pat. No. 5,958,792), biaryl amino acid amides (U.S. Pat. No. 5,948,696), thiophenes (U.S. Pat. No. 5,942,387), tricyclic Tetrahydroquinolines (U.S. Pat. No. 5,925,527), benzofurans (U.S. Pat. No. 5,919,955), isoquinolines (U.S. Pat. No. 5,916,899), hydantoin and thiohydantoin (U.S. Pat. No. 5,859,190), indoles (U.S. Pat. No. 5,856,496), imidazol-pyrido-indole and imidazol-pyrido-benzothiophenes (U.S. Pat. No. 5,856,107) substituted 2-methylene-2,3-dihydrothiazoles (U.S. Pat. No. 5,847,150), quinolines (U.S. Pat. No. 5,840,500), PNA (U.S. Pat. No. 5,831,014), containing tags (U.S. Pat. No. 5,721,099), polyketides (U.S. Pat. No. 5,712,146), morpholino-subunits (U.S. Pat. Nos. 5,698,685 and 5,506,337), sulfamides (U.S. Pat. No. 5,618,825), and benzodiazepines (U.S. Pat. No. 5,288,514).

As used herein combinatorial methods and libraries included traditional screening methods and libraries as well as methods and libraries used in interative processes.

b) Computer Assisted Drug Design

The disclosed Cdk2 and Rb can be used as targets for any molecular modeling technique to identify either the structure of the disclosed compositions or to identify potential or actual molecules, such as small molecules, which interact in a desired way with the disclosed compositions. It is understood that when using the disclosed compositions in modeling techniques, molecules, such as macromolecular molecules, will be identified that have particular desired properties such as inhibition of Rb phosphorylation or inhibition of Cdk2 activity. The molecules identified and isolated when using the disclosed compositions are also disclosed.

Thus, one way to isolate molecules that bind a molecule of choice is through rational design. This is achieved through structural information and computer modeling. Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

Examples of molecular modeling systems are the CHARMm and QUANTA programs, Polygen Corporation, Waltham, Mass. CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen, et al., 1988 Acta Pharmaceutica Fennica 97, 159-166; Ripka, New Scientist 54-57 (Jun. 16, 1988); McKinaly and Rossmann, 1989 Annu. Rev. Pharmacol. Toxiciol. 29, 111-122; Perry and Davies, QSAR: Quantitative Structure-Activity Relationships in Drug Design pp. 189-193 (Alan R. Liss, Inc. 1989); Lewis and Dean, 1989 Proc. R. Soc. Lond. 236, 125-140 and 141-162; and, with respect to a model enzyme for nucleic acid components, Askew, et al., 1989 J. Am. Chem. Soc. 111, 1082-1090. Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of molecules specifically interacting with specific regions of DNA or RNA, once that region is identified.

Although described above with reference to design and generation of compounds which could alter binding, one could also screen libraries of known compounds, including natural products or synthetic chemicals, and biologically active materials, including proteins, for compounds which alter substrate binding or enzymatic activity.

7. Compositions with Similar Funtions

It is understood that the compositions disclosed herein have certain functions, such as inhibiting Cdk2 activity or binding Cdk2 or Rb. Disclosed herein are certain structural requirements for performing the disclosed functions, and it is understood that there are a variety of structures which can perform the same function which are related to the disclosed structures, and that these structures will ultimately achieve the same result, for example inhibition of Cdk2 activity.

Throughout this application, various publications are referenced. The disclosures of these publications in their entireties are hereby incorporated by reference into this application in order to more fully describe the state of the art to which this invention pertains. The references disclosed are also individually and specifically incorporated by reference herein for the material contained in them that is discussed in the sentence in which the reference is relied upon.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

A number of cell cycle proteins are reexpressed in dying cardiac myocytes in the adult heart, particularly after ischemic injury (Reiss, K., et al. 1996. Experimental Cell Res. 225:44-54). Ischemia in vivo (Reiss, K., et al. 1996. Experimental Cell Res. 225:44-54) or hypoxia in vitro (Adachi, S., et al. 2001. Circ. Res. 88:408-414; Maejima, Y., et al. 2003. Cardiovasc. Res. 59:308-320) results in a rapid induction of Cdk2 activity. Some investigators have interpreted this finding as evidence of post-mitotic cardiac myocytes reentering the cell cycle and undergoing proliferative growth. Numerous stimuli unrelated to cell cycle progression induce Cdk2 activity and subsequently apoptosis such as irradiation (Anderson, J. A., et al. 1997. Mol. Biol. Cell 8:1195-1206), Tumor necrosis factor-α (Harvey, K. J., et al. 2000. J. Cell Biol. 148:59-72), Fas (Zhou, B. B., et al. 1998. Proc. Natl. Acad. Sci. U.S.A 95:6785-6790), staurosporine (Harvey, K. J., et al. 2000. J. Cell Biol. 148:59-72), hypoxia (Adachi, S., et al. 2001. Circ. Res. 88:408-414; Hauck, L., et al. 2002. Circ. Res. 91:782-789), and ischemia (Katchanov, J., et al. 2001. J. Neurosci. 21:5045-5053). Although Cdk2 activity is elevated during apoptosis, activation of the mitotic processes does not occur even in cells capable of proliferating (Zhou, B. B., et al. 1998. Proc. Natl. Acad. Sci. U.S.A 95:6785-6790). Likewise, interventions that inhibit cell cycle progression, without effecting Cdk2 activity, fail to block apoptosis (Hakem, A., et al. 1999. J. Exp. Med 189:957-968) supporting the notion that Cdk2 activation is not simply indicative of aberrant mitosis.

Increased Cdk2 activity has also been implicated in regulating apoptotic signaling pathways initiated by hypoxia in cardiac myocytes, at least in vitro (Adachi, S., et al. 2001. Circ. Res. 88:408-414; Hauck, L., et al. 2002. Circ. Res. 91:782-789). Increasing Cdk2 activity in cardiac myocytes by itself does not provoke cell cycle reentry (Akli, S., et al. 1999. Circ. Res. 85:319-328) but does appear to provoke apoptosis in vitro(Adachi, S., et al. 2001. Circ. Res. 88:408-414; Hauck, L., et al. 2002. Circ. Res. 91:782-789; Liao, H. S., et al. 2001. Circ. Res. 88:443-450). Alternatively, blocking Cdk2 activity in cultured myocytes inhibited apoptosis in response to hypoxia in vitro (Adachi, S., et al. 2001. Circ. Res. 88:408-414; Hauck, L., et al. 2002. Circ. Res. 91:782-789). Interestingly, a role for Cdk2 in cardiac preconditioning has also been reported. Pretreating cultured cardiac myocytes with NO donors attenuated hypoxia-induced elevations in Cdk2 activity by repressing Cyclin A (CycA) gene expression and promoting p21 accumulation (Maejima, Y., et al. 2003. Cardiovasc. Res. 59:308-320).

The mechanism whereby Cdk2 mediates its proapoptotic effect has been unclear in the art, but disclosed herein, are at least two potential mechanisms involving Rb- and p53, which are both capable of indirectly activating the mitochondrial pathway. Cdk2-mediated phosphorylation and stabilization of p53 leads to activation of Bax transcription, culminating in alterations of mitochondrial permeability transition (Hakem, A., et al. 1999. J. Exp. Med 189:957-968) and cytochrome-c release. Alternatively, inactivation of Rb is a critical step for the induction of apoptosis in many cell lines, whereas overexpression of Rb attenuates apoptosis induced by a variety of stimuli (Chau, B. N., et al. 2002. Nat. Cell Biol. 4:757-765; Wang, J., et al. 1997. Cancer Res 57:351-354; Ip, S. M., et al. 2001. Eur. J. Cancer 37:2475-2483) including hypoxia in cardiac myocytes (Hauck, L., et al. 2002. Circ. Res. 91:782-789; Wang, J., et al. 1997. Cancer Res 57:351-354). Germline disruption of Rb in mice leads to embryonic lethality in midgestation accompanied by apoptosis in a number of tissues including the central nervous system (CNS) (Jacks, T., et al. 1992. Nature. 359:295-300; Almasan, A., et al. 1995. Proc. Natl. Acad. Sci. U.S.A. 92:5436-5440; Clarke, A. R., et al. 1992. Nature 359:328-330; Macleod, K. F., et al. 1996. EMBO J 15:6178-6188; Wu, L., et al. 2003. Nature 421:942-947). This apoptosis required an additional cofactor, which in the case of CNS apoptosis, was thought to be hypoxia related to defective erythropoiesis (MacPherson, D., et al. 2003. Mol. Cell. Biol. 23:1044-1053). Rb can be inactivated through phosphorylation by kinases such as Cdk2 or cleavage by caspases, releasing free, transcriptionally active E2F (Janicke, R. U., et al. 1996. EMBO J. 15:6969-6978; Fattman, C. L., et al. 1997. J. Cell Biochem. 67:399-408). In vitro studies have suggested that hypoxia-induced Cdk2 activation in cardiac myocytes promotes apoptosis through phosphorylation of Rb and induction of E2F-dependent genes (Hauck, L., et al. 2002. Circ. Res. 91:782-789). E2F-1 regulates a number of genes involved in the apoptotic cascade including p53 family member, p73 (Pediconi, N., et al. 2003. Nat. Cell Biol. 5:552-558), Apaf-1 (Furukawa, Y., et al. 2002. J. Biol. Chem. 277:39760-39768; Moroni, M. C., et al. 2001. Nat. Cell Biol. 3:552-558), cytochrome c (Luciakova, K., et al. 2000. Biochem. J. 351:251-256) and caspase-3, -7, -8 and -9 (Nahle, Z., et al. 2002. Nat. Cell Biol. 4:859-864).

To determine the role of Cdk2 and Rb on myocardial I/R injury in vivo, a number of genetic mouse mutants and pharmacological inhibitors were utilized. The results demonstrate that Cdk2 activity is upregulated in vivo and attenuation of this activity prevents I/R injury. This effect is likely mediated through an Rb-dependent apoptotic pathway since loss of Rb in the heart significantly exacerbates I/R injury. These data indicate that the cell cycle proteins, Rb and Cdk2, are critical regulators of cardiac I/R injury in vivo and that Rb antagonizes the proapoptotic signals that accompany this stress.

a) Methods

(1) Mouse Strains and Genotyping.

Cdk2 deficient (Berthet, C., et al. 2003. Curr. Biol. 13:1775-1785), Rb^{MI} (Chau, B. N., et al. 2002. Nat. Cell Biol. 4:757-765) and cardiac-restricted Rb null mice (CRb^{L/L}) (MacLellan, W. R., et al. 2005. Mol. Cell. Biol. 25:2486-2497) have been described. Mice were maintained on a FVB background and littermate controls were used throughout the study. Genotypes of mice were determined by polymerase chain reaction as described (Agah, R., et al. 1997. J. Clin. Invest. 100:169-179).

(2) Myocardial Ischemia/Reperfusion Surgery and Infarct Size Analysis.

Male mice were subjected to myocardial ischemia/reperfusion as previously described (Wang, G., et al. 2005. Am. J. Physiol. Heart Circ. Physiol 288:H1290-H1295). Pentobarbital-anesthetized (50 mg/kg body wt i.p.) mice were intubated for positive pressure ventilation with oxygen-enriched room air during the surgical procedure. After left thoracotomy between ribs three and four, the pericardium was opened and a silk 8-0 suture was looped under the left anterior descending coronary artery 1-3 mm from the tip of the normally positioned left atrium. Ischemia was induced by ligation of the suture (a 1 to 2 mm section of PE-10 tubing was placed between the suture and the artery to prevent damage to the vessel). Rectal temperature was continuously measured and maintained at 36.5-37.5° C. Following a 30-min coronary artery occlusion, the suture was removed to allow coronary reperfusion followed by closure of the chest wall. After 24 hours of coronary artery reperfusion the heart was excised and postmortem perfused as previously described (Wang, G., et al. 2005. Am. J. Physiol. Heart Circ. Physiol 288:H1290-H1295). The infarct region was determined by perfusion with a 1% solution of 2,3,5-triphenyltetrazolium chloride (TTC) in phosphate buffer (pH 7.4, 37° C.). To delineate the risk region, the coronary artery was tied at the site of the previous occlusion and the aortic root perfused with a 1% solution of Evans blue dye. Infarct size was measured by planimetry with NIH Image and expressed as a percentage of the area at risk. For Cdk inhibitor studies, Roscovitine (Sigma) was dissolved in dimethyl sulfoxide (DMSO, Sigma) and mice were injected with 2.8 mg/gm intraperitoneally two hours prior to coronary ligations.

Limitation of IFS by pharmacological delayed preconditioning of the heart was induced with the nitric oxide donor diethylenetriamine/nitric oxide (DETA/NO) as previously reported (Wang, G., et al. 2005. Am. J. Physiol. Heart Circ. Physiol 288:H1290-H1295). After dissecting free the left jugular vein in pentobarbital-anesthetized WT mice, DETA/NO was given in 4 boluses 24-hours prior to 30-min coronary artery occlusion (0.1 mg/kg×4, i.v.).

(3) Cell Culture.

Neonatal rat cardiac myocytes were prepared as previously described (MacLellan, W. R., et al. 1994. J. Biol. Chem. 269:16754-16760). Cultured neonatal were serum-starved for 24 h prior to virus infection. Recombinant adenoviruses were constructed, propagated and titered as previously described (Graham, F. L. and Prevec, L. 1991. Methods in Molecular Biology).

(4) Western Blotting and Kinase Assays.

Western assays and Cdk kinase assays were performed as described (MacLellan, W. R., et al. 2005. Mol. Cell. Biol. 25:2486-2497), immunoprecipitating the specific kinase from mouse heart extracts. Western blotting was performed using the identical protein lysates. Antibodies were purchased from Santa Cruz Biotechnology (Santa Cruz). Protein expression was visualized using horseradish peroxidase-conjugated second antibodies and enhanced chemiluminescence reagents (Amersham Biosciences). Nonsaturated autoradiographs were quantitated with SigmaScan.

(5) Histology and Caspase Assays.

Hearts were either freshly frozen or fixed overnight in buffered 4% paraformaldehyde buffered and routinely processed. Evidence of apoptosis was assessed by detection of nuclear DNA fragmentation by terminal deoxynucleotidyl transferase-mediated dUTP nick end-labeling (Trevigen Inc.). At least 3,500 nuclei were examined per animal. Colorimetric caspase-3 assays were purchased from Promega and used according to the manufacturer's instructions.

(6) Statistical Analysis.

All data are presented as mean ± SEM. Results were compared by unpaired t-tests or analysis of variance with Fisher's PLSD tests, using significance at a P value <0.05.

b) Results

(1) Cdk2 Activity is Increased in Ischemia-reperfusion Injury and Induces Cardiac Myocyte Apoptosis.

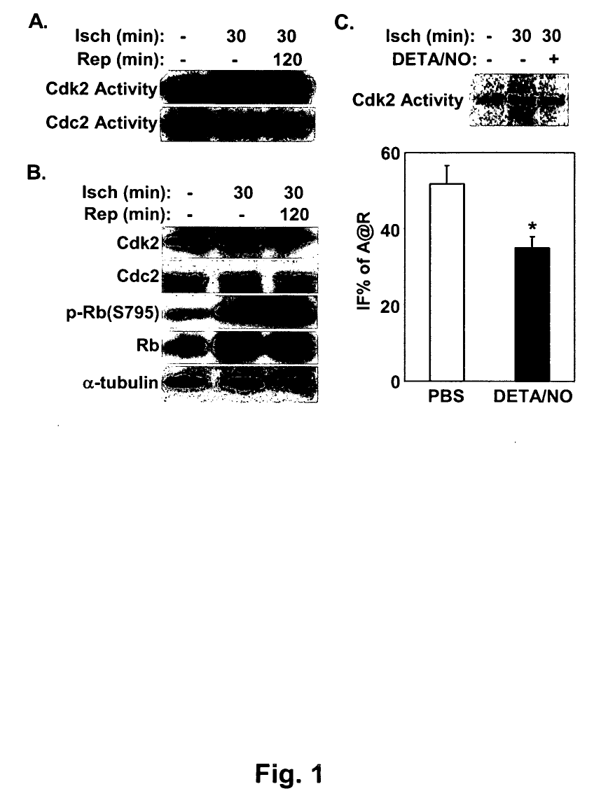

To determine if Cdk2 activity was upregulated in vivo in response to ischemia and/or reperfusion, adult mice were subjected to 30-min of myocardial ischemia via LAD occlusion with or without reperfusion. As shown in FIG. 1A, kinase assays to determine Cdk activity were performed on ventricular lysates. Cdk2 kinase activity was upregulated in ischemic ventricles and remained elevated during reperfusion despite. Interestingly, Cdc2 activity was also activated, although Cdk4 activation was not observed. To determine if the change in activity was related to changes in expression level and whether the increase in activity was associated with Rb phosphorylation, Western blots were performed on the same ventricular lysates. Levels of Cdk2 and Cdc2 were unchanged with ischemic injury (FIG. 1B). In contrast, levels of Rb increased along with its phosphorylated, inactivated form.

Nitric oxide (NO) is a signaling molecule that protects myocardium from I/R injury and may be important in mediating ischemic preconditioning. Studies in vitro have suggested that NO donors can attenuate hypoxia-induced increases in Cdk2 activity in cardiac myocytes, and reduce apoptotic cell death (Maejima, Y., et al. 2003. Cardiovasc. Res. 59:308-320). To determine if the activity of Cdk2 was regulated in vivo by NO donor-mediated preconditioning as well, wildtype mice were treated with a NO donor or vehicle, 24 hours before I/R injury. As shown in FIG. 1C, administration of a NO donor blocked the upregulation of Cdk2 seen with 30 minutes of ischemia, which was associated with a 32% reduction in infarct size when compared to vehicle treated mice (35.0±3.7 versus 51.8±4.8 IF % of the A@R; FIG. 1C, P<0.05). These data indicate that Cdk2 activity is regulated in I/R and correlates with myocardial injury.

(2) Cdk2 Induces Cardiac Myocyte Apoptosis.

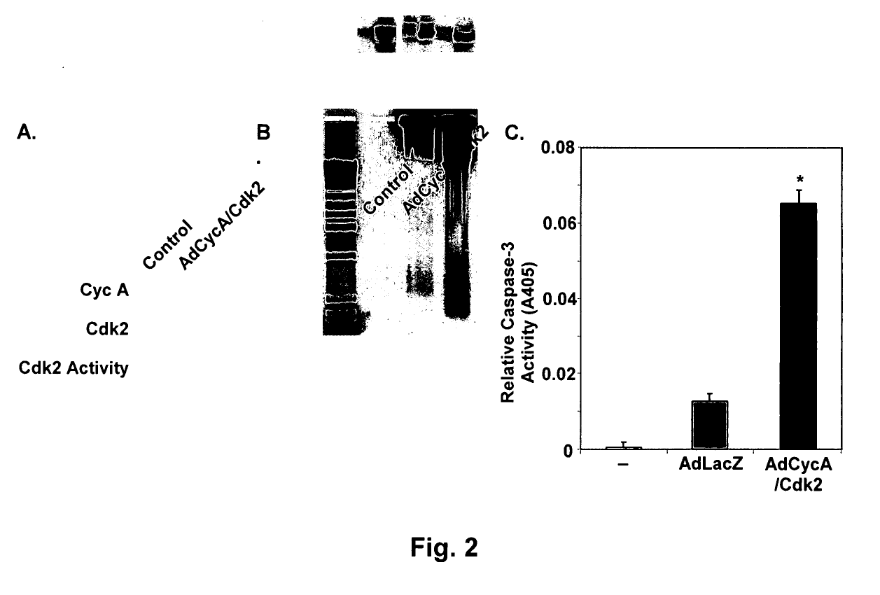

Inhibition of Cdk2 kinase activity in vitro has been shown to reduce cardiac myocyte apoptosis in response to hypoxia (Hauck, L., et al. 2002. Circ. Res. 91:782-789). To determine whether enhanced Cdk2 activity could also induce cardiac myocyte apoptosis, primary cultures of neonatal rat ventricular myocytes (NRVM) were infected with adenoviruses expressing either LacZ or CycA and Cdk2. As shown in FIG. 2A, overexpression of CycA and Cdk2 results in high-levels of Cdk2 kinase activity. This increased Cdk2 activity induces apoptosis in NRVM as documented by DNA laddering (FIG. 2B) and a 3.5-fold increase caspase-3 activity in AdCycA/Cdk2 versus AdLacZ infected NRVMs (P<0.001, FIG. 2C).

(3) Inhibition of Cdk2 Activity Attenuates Infarct Size in Vivo and Reduces Apoptosis.

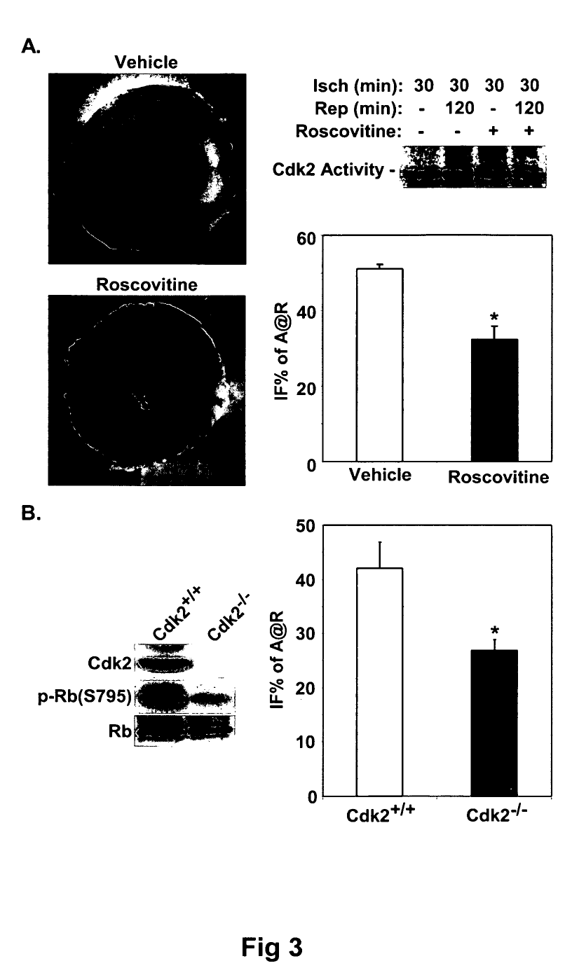

Although investigators have demonstrated that blocking Cdk2 activity in vitro in response to hypoxia attenuates cell death (Hauck, L., et al. 2002. Circ. Res. 91:782-789); whether this also protects cardiac myocytes in vivo was unknown. Thus, to test whether inhibition of Cdk2 activity would attenuate cell death in myocardium during I/R injury, mice were subjected to 30 min LAD coronary occlusion followed by 24 h of reperfusion. Mice were injected intraperitoneally two hours prior to ischemia with Roscovitine or vehicle (DMSO). Roscovitine is a well-characterized purine analogue that inhibits Cdk2 activity and has been used previously to inhibit Cdk2 activity in vivo (Golsteyn, R. M. 2005. Cancer Lett. 217:129-138). To ensure that Roscovitine was inhibiting the increase in Cdk2 activity observed in ischemic myocardium, Cdk2 kinase activity was measured in extracts prepared from ventricular tissue (FIG. 3A). Cdk2 complexes were immunoprecipitated from ventricular lysates from each of the indicated treatment groups. Treatment with Roscovitine inhibited the expected increase in Cdk2 activity. This inhibition of Cdk2 led to a 36.7% reduction in infarct size in the Roscovitine treated mice when compared to vehicle treated mice (34.2±2.6 versus 51.4±2.2 IFS % of A@R, P<0.001, FIG. 3A).

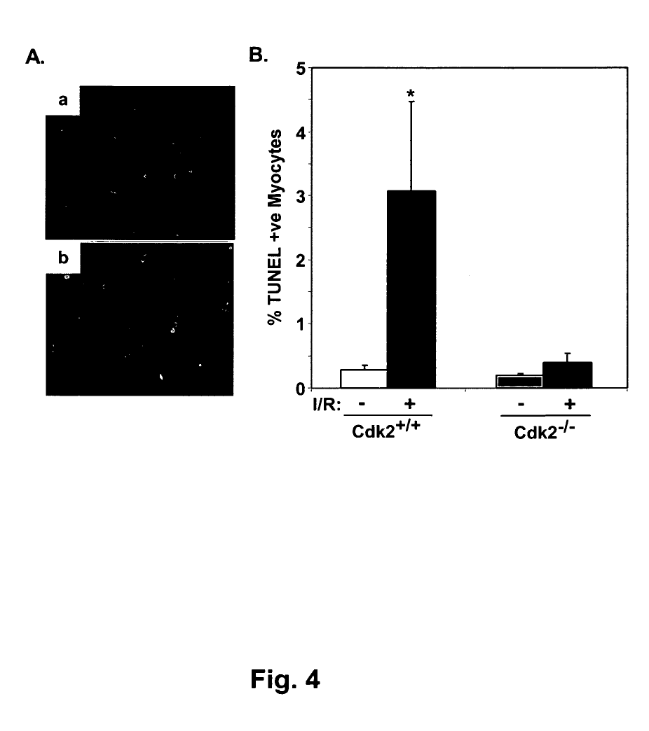

Although these data indicate that Cdk2 is a critical regulator of cardiac myocyte injury in vivo in response to I/R, Roscovitine has been reported to inhibit the activity of Cdc2, albeit at a lower affinity. Therefore, to confirm that this reduction in infarct size was specifically related to decreased Cdk2 kinase activity, Cdk2-defient mice were tested by subjecting them to I/R injury. Cdk2-deficient mice had a 36.1% reduction in infarct size when compared to littermate controls (26.9±2.0 versus 42.1±4.8 IFS % of A@R, P<0.05, FIG. 3B). To determine if inhibition of Cdk2 activity attenuates I/R-induced apoptosis in vivo, the number of TUNEL positive nuclei in the infarct border zone was determined in Cdk2^{+/+} and Cdk2^{-/-} mice. The relative number of TUNEL positive nuclei in the border zone was 7.8-fold higher in Cdk2^{+/+} ventricles when compared to Cdk2^{-/-} hearts after I/R injury (3.08±1.4 versus 0.39±0.14% TUNEL positive nuclei; P=0.05, FIG. 4A & B).

(4) Rb is Cardioprotective in Ischemia-reperfusion Injury.

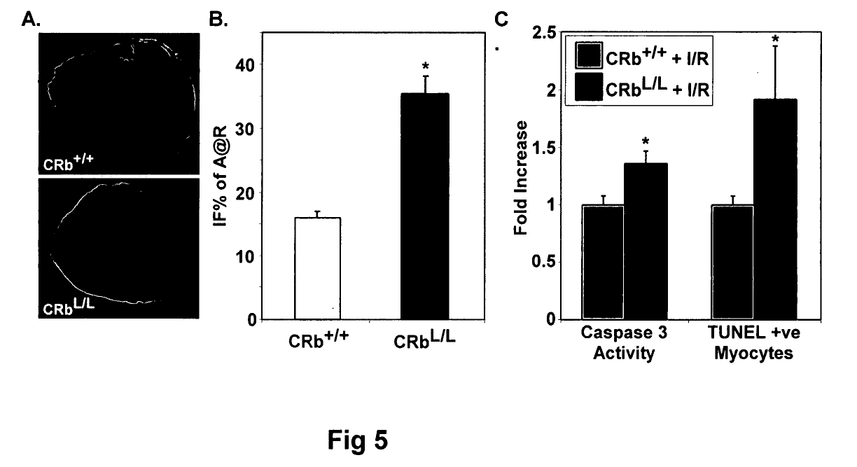

Rb is one of the primary substrate of Cdk2 kinase activity during the cell cycle, although its role as a Cdk2-target in apoptosis signaling pathways is less clear. The data demonstrate that Rb is phosphorylated and presumably inactivated in ischemic wildtype myocardium (FIG. 1A) but not in myocardium deficient for Cdk2 (FIG. 3B). To determine if Rb is an important target for Cdk2-dependent cell death in the heart, cardiac-restricted Rb-deficient mice were subjected to I/R injury (MacLellan, W. R., et al. 2005. Mol. Cell. Biol. 25:2486-2497). These mice appeared phenotypically and biochemically normal at baseline. I/R injury resulted in a 2.4-fold increase in IFS in CRb^{L/L} mice when compared to CRb^{+/+} controls (37.2+4.9% versus 15.5+1.1%, P<0.001, FIG. 5A & B), demonstrating significant exacerbation of ischemic injury in CRb^{L/L} mice. To explore possible mechanisms underlying the enhanced injury seen in CRb^{L/L} mice after I/R, the number of TUNEL positive nuclei was determined in CRb^{+/+} versus CRb^{L/L} ventricles after ischemic injury to determine if apoptosis might be altered. At baseline there were no significant differences in TUNEL positive nuclei between genotypes. However, the relative number of TUNEL positive myocyte nuclei in the border zone was increased in CRb^{L/L} ventricles when compared to CRb^{+/+} hearts after ischemic injury (1.92±0.46 versus 1.0±0.08-fold; P<0.05). Caspase-3 activity was also augmented in ischemic myocardium from CRb^{L/L} compared to that from CRb^{+/+} mice (1.36±0.11 versus 1.0±0.08-fold; P<0.05). Thus, given the extensive data implicating the Cdk2-Rb-E2F pathway in regulating apoptosis, the increased infarct size represents an increased susceptibility to apoptosis.

(5) Caspase-resistant Rb is not Cardioprotective in Vivo.

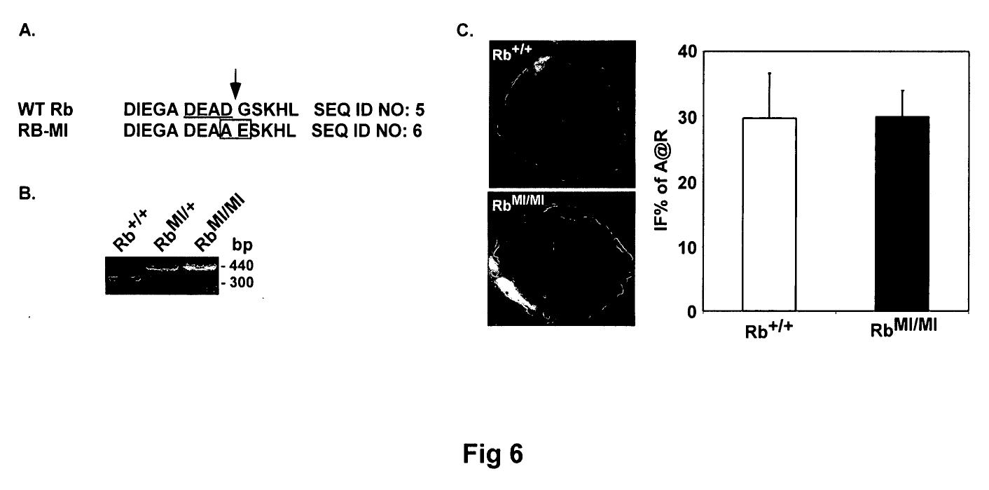

Although Rb is functionally inactivated by Cdk2-phosphorylation, its function can also be blocked by caspase cleavage during apoptosis (Tan, X. and Wang, J. Y. 1998. Trends Cell Biol. 8:116-120). To assess the relative role of caspase-dependent inactivation of Rb in cardiac myocyte ischemic injury, the effects of I/R was determined in caspase-resistant Rb mutant mice (Rb-MI) (Chau, B. N., et al. 2002. Nat. Cell Biol. 4:757-765). These mice have been engineered with a mutated Rb gene where the Asp-Glu-Ala-Asp-Gly of the caspase cleavage site shown in SEQ ID NO. 5 has been converted to Asp-Glu-Ala-Ala-Glu as shown in SEQ ID NO. 6 and then “knocked-in” to the Rb locus (FIG. 6A & B). This mutation prevents cleavage and inactivation of Rb by caspases. Rb-MI mice are viable and appear phenotypically normal at baseline but are resistant to some (TNFα) but not all (doxorubicin) inducers of apoptosis. Wildtype or Rb-MI littermates were subjected to an I/R and the extent of myocardial injury quantified. As shown, infarct size was indistinguishable between the two genotypes (29.7±7.0 vs. 29.9±4.1; P=n.s., FIG. 6C). These data indicate that inactivation of Rb by caspase cleavage does not play a significant role in mediating cardiac injury in response to I/R.

(6) Bax Upregulation is Exaggerated in Rb-null Myocytes with ischemia.

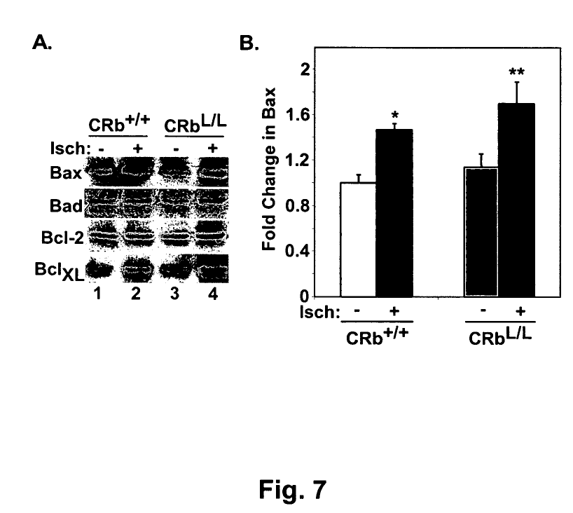

To determine if the susceptibility of Rb-deficient myocytes to apoptosis in response to I/R injury in vivo was related to altered expression of apoptotic regulatory proteins, Bcl-2, Bcl_{XL}, Bax and Bad protein expression was examined in total ventricular lysates from CRb^{+/+} or CRb^{L/L} mice at baseline and after ischemia (FIG. 7A). There were no statistically significant differences in expression of any apoptotic proteins at baseline in CRb^{+/+} versus CRb^{L/L} hearts (Lane 1 versus 3). Expression of Bcl-2 and Bad did not change significantly in CRb^{+/+}versus CRb^{L/L} hearts after 30 minutes of ischemia (Lane 2 versus 4). Levels of Bcl_{XL} increased in both CRb^{+/+} (1.0±0.07 versus 1.47±0.05; P<0.05) and CRb^{L/L} (1.18±0.11 versus 1.7±0.19; P<0.05) hearts after thirty minutes of ischemia; however, there was no difference in the levels when lysates from ischemic CRb^{+/+} versus CRb^{L/L} myocardium were compared. In contrast, levels of Bax were significantly increased in ischemic CRb^{L/L} versus CRb^{+/+} hearts (1.62±0.24 versus 1.15±0.07; P<0.05, FIG. 7B).

c) Discussion

Growing evidence suggests that cell cycle proteins, and Cdk2 activity in particular, are reexpressed in dying cells and play a central role in the regulation of programmed cell death (Golsteyn, R. M. 2005. Cancer Lett. 217:129-138; Jin, Y. H., et al. 2003. Biochem. Biophys. Res. Commun. 305:974-980; Osuga, H., et al. 2000. Proc. Natl. Acad. Sci. U.S.A. 97:10254-10259). The data presented herein establishes a similar role for Cdk2 and its downstream targets in the regulation of myocardial I/R injury. It has been appreciated that myocardial ischemia in vivo (Reiss, K., et al. 1996. Experimental Cell Res. 225:44-54) or hypoxia in vitro (Adachi, S., et al. 2001. Circ. Res. 88:408-414; Maejima, Y., et al. 2003. Cardiovasc. Res. 59:308-320) results in an induction of Cdk2 activity but this has often been interpreted as evidence of cell cycle reentry in cardiac myocytes. The ability of preconditioning stimuli (here, NO) to modulate Cdk2 activity in ischemic myocardium argues against a simple role for this kinase in cardiac myocyte proliferation. Although increasing Cdk2 activity is sufficient to promote proliferation in many cell types, this does not seem to be the case in cardiac myocytes, as increasing Cdk2 activity in cultured cardiac myocytes was ineffective to provoke S phase entry (Akli, S., et al. 1999. Circ. Res. 85:319-328). This is consistent with studies demonstrating that enhanced Cdk2 activity could promote apoptotic cell death even when cell cycle reentry was blocked (Hakem, A., et al. 1999. J. Exp. Med 189:957-968). Thus, given the extensive data implicating the Cdk2-Rb-E2F pathway in regulating apoptosis, it is likely that the effects of the interventions described herein on infarct size represents a modulation of apoptosis in the heart.

Until recently, it was believed that Cdk2 and its activator CycE play essential roles in the progression of the cell cycle. However, studies using knockout mouse models revealed that neither Cdk2 (Berthet, C., et al. 2003. Curr. Biol. 13:1775-1785; Ortega, S., et al. 2003. Nat. Genet. 35:25-31) nor CycE (Geng, Y., et al. 2003. Cell 114:431-443; Parisi, T., et al. 2003. EMBO J. 22:4794-4803) are essential in vivo. Loss of Cdk2 had a relatively minor effect on progression through the mitotic cell cycle, phenotypically manifesting only as delayed entry into S phase. It has been realized that this data is consistent with Cdk2 playing a critical role in cell death signaling pathways, at least those initiated by I/R, in spite of the fact that Cdk2 appears to be dispensable for normal cell cycle progression (Berthet, C., et al. 2003. Curr. Biol. 13:1775-1785). This is the first in vivo evidence for a direct role of Cdk2 in cell death pathways in the heart. The mechanisms by which re-activation of Cdk2 in postmitotic cardiac myocytes could propagate an apoptotic signal to the cell death machinery are poorly understood. The primary phosphorylation target of Cdk2 kinase, at least in cell cycle progression, is the retinoblastoma protein (Rb). The data demonstrating Rb is phosphorylated in ischemic wildtype but not Cdk2-null myocardium indicates that Rb is one of the main targets of Cdk2 in I/R as well. At least one report has suggested that hypoxia-induced Cdk2 activation promotes cardiac myocyte apoptosis through induction of E2F-dependent genes (Hauck, L., et al. 2002. Circ. Res. 91:782-789). However, Rb may not be the only target of Cdk2 during ischemia. Cdk2 has also been implicated in mediating p53-dependent cell death pathways. Studies in DNA damaged cells demonstrated that serine 315 of p53 can be phosphorylated by Cdk2, leading to stabilization of the protein and preservation of its transcriptional activity (Price, B. D., et al. 1995. Oncogene 11:73-80).

Cdk2-mediated p53 phosphorylation led to activation of Bax transcription, culminating in alterations of mitochondrial permeability (Hakem, A., et al. 1999. J. Exp. Med 189:957-968). In other systems, inhibition of Cdk2 has been shown to prevent mitochondrial permeability transition and caspase activation (Hakem, A., et al. 1999. J. Exp. Med 189:957-968; Jin, Y. H., et al. 2003. Biochem. Biophys. Res. Commun. 305:974-980) but whether this is a direct effect on the function of the mitochondrial permeability pore or an indirect effect remains to be clarified. Likewise, despite p53's fundamental role in regulating apoptosis in other systems, its function in the heart is unclear. Although p53 expression levels are increased in hypoxic cultured cardiac myocytes (Long, X., et al. 1997. J. Clin. Invest. 99:2635-2643) and ischemic myocardium in vivo (Maulik, N., et al. 2000. FEBS Lett. 485:7-12), ischemic injury was unchanged in p53 null mice (Bialik, S., et al. 1997. J. Clin. Invest 100:1363-1372; Xie, Z., et al. 2000. Jpn. J. Physiol. 50:159-162). Myocyte apoptosis occurs with the same frequently in ischemic hearts of p53-null mice as in wild-type littermates indicating the existence of p53-independent pathways, possibly Rb-E2F dependent, that mediate myocyte apoptosis during myocardial infarction. Pharmacological inhibitors of Cdk2 have been developed for cancer treatment (Golsteyn, R. M. 2005. Cancer Lett. 217:129-138; Hirai, H., et al. 2005. Curr. Top. Med. Chem. 5:167-179). It has now been discovered as describe herein that targeting Cdk2 activity in clinical situations where apoptotic pathways are activated such as myocardial infarction is proven to be beneficial.

Although Rb has been implicated in the regulation of apoptosis, this is the first study to demonstrate a susceptibility to myocardial I/R injury in vivo. Widespread programmed cell death was seen with germline deletion of Rb (Macleod, K. F., et al. 1996. EMBO J 15:6178-6188), but this was related to its role in extraembryonic lineages since neurogenesis and erythropoiesis was normal and apoptosis restricted with a wildtype placenta (Wu, L., et al. 2003. Nature 421:942-947). Central nervous system (CNS)-specific excision of Rb in vivo did not provoke significant apoptosis (MacPherson, D., et al. 2003. Mol. Cell. Biol. 23:1044-1053; Marino, S., et al. 2000. Genes Dev. 14:994-1004; de Bruin, A., et al. 2003. Proc. Natl. Acad. Sci. U.S.A. 100:6546-6551). Apoptosis seen in germline deletions of Rb was postulated to require an additional cofactor, which in the case of CNS apoptosis, was thought to be hypoxia (MacPherson, D., et al. 2003. Mol. Cell. Biol. 23:1044-1053). Overexpression of Rb can also attenuate hypoxia-induced apoptosis in cardiac myocytes (Hauck, L., et al. 2002. Circ. Res. 91:782-789). Therefore, Rb's normal physiological role in the heart might be to prevent apoptosis in response to environmental stressors. While Rb is postulated to inhibit apoptosis in many cell culture systems, this is the first evidence directly implicating Rb in cell survival in adult post-mitotic myocardium. The present study demonstrates that cardiac-specific Rb-deficient mice are more susceptible to myocardial I/R injury in vivo.

It was observed herein that Rb is phosphorylated on a known Cdk2 site, Ser-795 during myocardial ischemia. This site was also phosphorylated during neuronal ischemia; however, there elevated Rb phosphorylation was dependent upon Cdk4 activity (Rashidian, J., et al. 2005. Proc. Natl. Acad. Sci. U.S.A. 102:14080-14085). The studies exclude the possibility that caspase-dependent inactivation of Rb plays a major role in the setting of I/R injury, since mice with a mutated Rb gene preventing it from being cleaved by caspases showed similar IFS as compared to wildtype mice. This contrasts with the increased susceptibility of Rb-MI mice to endotoxin-induced apoptosis (Chau, B. N., et al. 2002. Nat. Cell Biol. 4:757-765). Although, even in this model, the protective effects of Rb-MI were tissue specific indicating that the mechanism of inactivation of Rb with apoptotic inducers may be both signal- and cell type-dependent.

In summary, it has been discovered that Rb is a critical protective molecule in the heart and that Cdk2, most likely through phosphorylation of Rb, is a key regulator of myocardial I/R injury. Furthermore, it has been discovered that the Cdk2-Rb signaling module plays an important role in regulating myocardial I/R injury through apoptotic pathways. Accordingly, Rb's normal physiological role in the heart might be to prevent apoptosis in response to environmental stressors such as ischemia. These discoveries should prove useful to identify the mechanism(s) underlying Rb's anti-apoptotic effect and the role of E2F activity in this process. Apoptosis is known to play a pivotal role in a number of cardiovascular diseases (MacLellan, W. R. and Schneider, M. D. 1997. Circ. Res. 81:137-144) and it will be critical in the future to determine the role that cell cycle regulators play in this process.

F. REFERENCES
Adachi, S., Ito, H., Tamamori-Adachi, M., Ono, Y., Nozato, T., Abe, S., Ikeda, M., Marumo, F., and Hiroe, M. 2001. Cyclin A/cdk2 activation is involved in hypoxia-induced apoptosis in cardiomyocytes. Circ. Res. 88:408-414.
Agah, R., Frenkel, P. A., French, B. A., Michael, L. H., Overbeek, P. A., and Schneider, M. D. 1997. Gene recombination in postmitotic cells. Targeted expression of Cre recombinase provokes cardiac-restricted, site-specific rearrangement in adult ventricular muscle in vivo. J. Clin. Invest. 100:169-179.
Akli, S., Zhan, S., Abdellatif, M., and Schneider, M. D. 1999. E1A can provoke G1 exit that is refractory to p21 and independent of activating cdk2. Circ. Res. 85:319-328.
Almasan, A., Yin, Y., Kelly, R. E., Lee, E. Y. H. P., Bradley, A., Li, W., Bertino, J. R., and Wahl, G. M. 1995. Deficiency of retinoblastoma protein leads to inappropriate s-phase entry, activation of E2F-responsive genes, and apoptosis. Proc. Natl. Acad. Sci. U.S.A. 92:5436-5440.
Anderson, J. A., Lewellyn, A. L., and Maller, J. L. 1997. Ionizing radiation induces apoptosis and elevates cyclin A1-Cdk2 activity before but not after the midblastula transition in Xenopus. Mol. Biol. Cell 8:1195-1206.
Bell, L. A. and Ryan, K. M. 2004. Life and death decisions by E2F-1. Cell Death Differ. 11:137-142.
Berthet, C., Aleem, E., Coppola, V., Tessarollo, L., and Kaldis, P. 2003. Cdk2 knockout mice are viable. Curr. Biol. 13:1775-1785.
Bialik, S., Geenen, D. L., Sasson, I. E., Cheng, R., Homer, J. W., Evans, S. M., Lord, E. M., Koch, C. J., and Kitsis, R. N. 1997. Myocyte apoptosis during acute myocardial infarction in the mouse localizes to hypoxic regions but occurs independently of p53. J. Clin. Invest 100:1363-1372.
Bouchard, C., Thieke, K., Maier, A., Saffrich, R., Hanley-Hyde, J., Ansorge, W., Reed, S., Sicinski, P., Bartek, J., and Eilers, M. 1999. Direct induction of cyclin D2 by Myc contributes to cell cycle progression and sequestration of p27. EMBO J 18:5321-5333.
Chau, B. N., Borges, H. L., Chen, T. T., Masselli, A., Hunton, I. C., and Wang, J. Y. 2002. Signal-dependent protection from apoptosis in mice expressing caspase-resistant Rb. Nat. Cell Biol. 4:757-765.
Clarke, A. R., Maandag, E. R., van Roon, M., van der Lugt, N. M., van der Valk, M., Hooper, M. L., Berns, A., and te, R. H. 1992. Requirement for a functional Rb-1 gene in murine development. Nature 359:328-330.
de Bruin, A., Wu, L., Saavedra, H. I., Wilson, P., Yang, Y., Rosol, T. J., Weinstein, M., Robinson, M. L., and Leone, G. 2003. Rb function in extraembryonic lineages suppresses apoptosis in the CNS of Rb-deficient mice. Proc. Natl. Acad. Sci. U.S.A. 100:6546-6551.
Fattman, C. L., An, B., and Dou, Q. P. 1997. Characterization of interior cleavage of retinoblastoma protein in apoptosis. J. Cell Biochem. 67:399-408.
Furukawa, Y., Nishimura, N., Furukawa, Y., Satoh, M., Endo, H., Iwase, S., Yamada, H., Matsuda, M., Kano, Y., and Nakamura, M. 2002. Apaf-1 is a mediator of E2F-1-induced apoptosis. J. Biol. Chem. 277:39760-39768.
Geng, Y., Yu, Q., Sicinska, E., Das, M., Schneider, J. E., Bhattacharya, S., Rideout, W. M., Bronson, R. T., Gardner, H., and Sicinski, P. 2003. Cyclin E ablation in the mouse. Cell 114:431-443.
Golsteyn, R. M. 2005. Cdk1 and Cdk2 complexes (cyclin dependent kinases) in apoptosis: a role beyond the cell cycle. Cancer Lett. 217:129-138.
Graham, F. L. and Prevec, L. 1991. Methods in Molecular Biology, vol.7. The Humana Press Inc., Clifton, N.J.
Hakem, A., Sasaki, T., Kozieradzki, I., and Penninger, J. M. 1999. The cyclin-dependent kinase Cdk2 regulates thymocyte apoptosis. J. Exp. Med 189:957-968.
Harvey, K. J., Lukovic, D., and Ucker, D. S. 2000. Caspase-dependent Cdk activity is a requisite effector of apoptotic death events. J. Cell Biol. 148:59-72.
Hauck, L., Hansmann, G., Dietz, R., and von Harsdorf, R. 2002. Inhibition of hypoxia-induced apoptosis by modulation of retinoblastoma protein-dependent signaling in cardiomyocytes. Circ. Res. 91:782-789.
Hirai, H., Kawanishi, N., and Iwasawa, Y. 2005. Recent advances in the development of selective small molecule inhibitors for cyclin-dependent kinases. Curr. Top. Med. Chem. 5:167-179.
Hou, S. T., Callaghan, D., Fournier, M. C., Hill, I., Kang, L., Massie, B., Morley, P., Murray, C., Rasquinha, I., Slack, R. et al. 2000. The transcription factor E2F1 modulates apoptosis of neurons. J. Neurochem. 75:91-100.
Ip, S. M., Huang, T. G., Yeung, W. S., and Ngan, H. Y. 2001. pRb-expressing adenovirus Ad5-Rb attenuates the p53-induced apoptosis in cervical cancer cell lines. Eur. J. Cancer 37:2475-2483.
Jacks, T., Fazeli, A., Schmitt, E. M., Bronson, R. T., Goodell, M. A., and Weinberg, R. A. 1992. Effects of an Rb mutation in the mouse. Nature. 359:295-300.

Janicke, R. U., Walker, P. A., Lin, X. Y., and Porter, A. G. 1996. Specific cleavage of the retinoblastoma protein by an ICE-like protease in apoptosis. EMBO J. 15:6969-6978.
Jin, Y. H., Yim, H., Park, J. H., and Lee, S. K. 2003. Cdk2 activity is associated with depolarization of mitochondrial membrane potential during apoptosis. Biochem. Biophys. Res. Commun. 305:974-980.
Katchanov, J., Harms, C., Gertz, K., Hauck, L., Waeber, C., Hirt, L., Priller, J., von Harsdorf, R., Bruck, W., Hortnagl, H. et al. 2001. Mild cerebral ischemia induces loss of cyclin-dependent kinase inhibitors and activation of cell cycle machinery before delayed neuronal cell death. J. Neurosci. 21:5045-5053.
Liao, H. S., Kang, P. M., Nagashima, H., Yamasaki, N., Usheva, A., Ding, B., Lorell, B. H., and Izumo, S. 2001. Cardiac-specific overexpression of cyclin-dependent kinase 2 increases smaller mononuclear cardiomyocytes. Circ. Res. 88:443-450.
Long, X., Boluyt, M. O., Hipolito, M. L., Lundberg, M. S., Zheng, J. S., O'Neill, L., Cirielli, C., Lakatta, E. G., and Crow, M. T. 1997. p53 and the hypoxia-induced apoptosis of cultured neonatal rat cardiac myocytes. J. Clin. Invest. 99:2635-2643.
Luciakova, K., Barath, P., Li, R., Zaid, A., and Nelson, B. D. 2000. Activity of the human cytochrome cl promoter is modulated by E2F. Biochem. J. 351:251-256.
MacLellan, W. R. and Schneider, M. D. 1997. Death by design. Programmed cell death in cardiovascular biology and disease. Circ. Res. 81:137-144.
MacLellan, W. R., Garcia, A., Oh, H., Frenkel, P., Jordan, M. C., Roos, K. P., and Schneider, M. D. 2005. Overlapping roles of pocket proteins in the myocardium are unmasked by germ line deletion of p130 plus heart-specific deletion of Rb. Mol. Cell. Biol. 25:2486-2497.
MacLellan, W. R., Lee, T. C., Schwartz, R. J., and Schneider, M. D. 1994. Transforming growth factor-β response elements of the skeletal α-actin gene. J. Biol. Chem. 269:16754-16760.
Macleod, K. F., Hu, Y., and Jacks, T. 1996. Loss of Rb activates both p53-dependent and independent cell death pathways in the developing mouse nervous system. EMBO J 15:6178-6188.
MacManus, J. P., Koch, C. J., Jian, M., Walker, T., and Zurakowski, B. 1999. Decreased brain infarct following focal ischemia in mice lacking the transcription factor E2F1. Neuroreport. 10:2711-2714.
MacPherson, D., Sage, J., Crowley, D., Trumpp, A., Bronson, R. T., and Jacks, T. 2003. Conditional mutation of Rb causes cell cycle defects without apoptosis in the central nervous system. Mol. Cell. Biol. 23:1044-1053.
Maejima, Y., Adachi, S., Ito, H., Nobori, K., Tamamori-Adachi, M., and Isobe, M. 2003. Nitric oxide inhibits ischemia/reperfusion-induced myocardial apoptosis by modulating cyclin A-associated kinase activity. Cardiovasc. Res. 59:308-320.
Marino, S., Vooijs, M., van Der, G. H., Jonkers, J., and Berns, A. 2000. Induction of medulloblastomas in p53-null mutant mice by somatic inactivation of Rb in the external granular layer cells of the cerebellum. Genes Dev. 14:994-1004.
Maulik, N., Sasaki, H., Addya, S., and Das, D. K. 2000. Regulation of cardiomyocyte apoptosis by redox-sensitive transcription factors. FEBS Lett. 485:7-12.
Moroni, M. C., Hickman, E. S., Denchi, E. L., Caprara, G., Colli, E., Cecconi, F., Muller, H., and Helin, K. 2001. Apaf-1 is a transcriptional target for E2F and p53. Nat. Cell Biol. 3:552-558.
Nahle, Z., Polakoff, J., Davuluri, R. V., McCurrach, M. E., Jacobson, M. D., Narita, M., Zhang, M. Q., Lazebnik, Y., Bar-Sagi, D., and Lowe, S. W. 2002. Direct coupling of the cell cycle and cell death machinery by E2F. Nat. Cell Biol. 4:859-864.
Ortega, S., Prieto, I., Odajima, J., Martin, A., Dubus, P., Sotillo, R., Barbero, J. L., Malumbres, M., and Barbacid, M. 2003. Cyclin-dependent kinase 2 is essential for meiosis but not for mitotic cell division in mice. Nat. Genet. 35:25-31.
Osuga, H., Osuga, S., Wang, F., Fetni, R., Hogan, M. J., Slack, R. S., Hakim, A. M., Ikeda, stroke. Proc. Natl. Acad. Sci. U.S.A. 97:10254-10259.
Parisi, T., Beck, A. R., Rougier, N., McNeil, T., Lucian, L., Werb, Z., and Amati, B. 2003. Cyclins E1 and E2 are required for endoreplication in placental trophoblast giant cells. EMBO J. 22:4794-4803.
Pediconi, N., Ianari, A., Costanzo, A., Belloni, L., Gallo, R., Cimino, L., Porcellini, A., Screpanti, I., Balsano, C., Alesse, E. et al. 2003. Differential regulation of E2F1 apoptotic target genes in response to DNA damage. Nat. Cell Biol. 5:552-558.
Perez-Roger, I., Kim, S. H., Griffiths, B., Sewing, A., and Land, H. 1999. Cyclins D1 and D2 mediate myc-induced proliferation via sequestration of p27(Kip1) and p21 (Cip1). EMBO J. 18:5310-5320.
Price, B. D., Hughes-Davies, L., and Park, S. J. 1995. Cdk2 kinase phosphorylates serine 315 of human p53 in vitro. Oncogene 11:73-80.
Rashidian, J., Iyirhiaro, G., Aleyasin, H., Rios, M., Vincent, I., Callaghan, S., Bland, R. J., Slack, R. S., During, M. J., and Park, D. S. 2005. Multiple cyclin-dependent kinases signals are critical mediators of ischemia/hypoxic neuronal death in vitro and in vivo. Proc. Natl. Acad. Sci. U.S.A. 102:14080-14085.
Reiss, K., Cheng, W., Giorando, A., DeLuca, A., Li, B., Kajatura, J., and Anversa, P. 1996. Myocardial infarction is coupled with activation of cyclin and cyclin-dependent kinases in myocytes. Experimental Cell Res. 225:44-54.
Tan, X. and Wang, J. Y. 1998. The caspase-RB connection in cell death. Trends Cell Biol. 8:116-120.
Wang, G., Liem, D. A., Vondriska, T. M., Honda, H. M., Korge, P., Pantaleon, D. M., Qiao, X., Wang, Y., Weiss, J. N., and Ping, P. 2005. Nitric oxide donors protect murine myocardium against infarction via modulation of mitochondrial permeability transition. Am. J. Physiol. Heart Circ. Physiol 288:H1290-H1295. Wang, J., Guo, K., Wills, K. N., and Walsh, K. 1997. Rb functions to inhibit apoptosis during myocyte differentiation. Cancer Res 57:351-354.
Webster, K. A., Discher, D. J., Kaiser, S., Hernandez, O., Sato, B., and Bishopric, N. H. 1999. Hypoxia-activated apoptosis of cardiac myocytes requires reoxygenation or a pH shift and is independent of p53. J. Clin. Invest 104:239-252.
Wu, L., de Bruin, A., Saavedra, H. I., Starovic, M., Trimboli, A., Yang, Y., Opavska, J., Wilson, P., Thompson, J. C., Ostrowski, M. C. et al. 2003. Extra-embryonic function of Rb is essential for embryonic development and viability. Nature 421:942-947.
Xie, Z., Koyama, T., Abe, K., Fuji, Y., Sawa, H., and Nagtashima, K. 2000. Upregulation of P53 protein in rat heart subjected to a transient occlusion of the coronary artery followed by reperfusion. Jpn. J. Physiol. 50:159-162.
Yamasaki, L., Bronson, R., Williams, B. O., Dyson, N. J., Harlow, E., and Jacks, T. 1998. Loss of E2F-1 reduces tumorigenesis and extends the lifespan of Rb1(+/−)mice. Nat. Genet 18:360-364.
Zhou, B. B., Li, H., Yuan, J., and Kirschner, M. W. 1998. Caspase-dependent activation of cyclin-dependent kinases during Fas-induced apoptosis in Jurkat cells. Proc. Natl. Acad Sci. U.S.A 95:6785-6790.

G. Sequences
1. SEQ ID NO: 1 Cyclin dependent kinase 2 (Cdk2) nucleotide sequence Genbank Accession No. AF512553
2. SEQ ID NO: 2 Cyclin dependent kinase 2 (Cdk2) amino acid sequence Genbank Accession No. AAM34794
3. SEQ ID NO: 3 Retinoblastoma nucleic acid sequence Genbank Accession No. M28419
4. SEQ ID NO: 4 Retinoblastoma amino acid sequence Genbank Accession No. AAA69808
5. SEQ ID NO: 5 DIEGADEADGSKHL
6. SEQ ID NO: 6 DIEGADEAAESKHL

What is claimed is:

1. A method of reducing ischemia/reperfusion injury in a subject in need thereof comprising administering to the subject an agent that inhibits cyclin dependent kinase 2 (Cdk2) activity.

2. The method of claim 1, wherein the ischemia/reperfusion injury occurs following an ischemia/reperfusion event selected from the group consisting of myocardial ischemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypoxia, retinal ischemia, renal ischemia, ischemia/reperfusion events occurring during cardiac surgery where a heart lung machine is used such as Coronary artery bypassing, and ischemia/reperfusion events occurring during the preservation of organs for transplant.

3. The method of claim 1, wherein the agent is administered after the ischemia/reperfusion event.

4. The method of claim 3, wherein the agent is administered within 30 minutes, 1, 2, 6, 12, or 24 hour(s) following the ischemia/reperfusion event.

5. The method of claim 1, wherein the agent is administered before the ischemia/reperfusion event.

6. The method of claim 5, wherein the agent is administered at least 1, 2, 6, 12, or 24 hour(s) before the ischemia/reperfusion event.

7. The method of claim 5, wherein the agent is administered at least 2 days, 3 days, 1 week, or 2 weeks before the ischemia/reperfusion event.

8. The method of claim 1, wherein the agent is administered during the ischemia/reperfusion event.

9. The method of claim 1, wherein the agent is selected from the group consisting of purine analogs or derivatives thereof, pyrimidine analogs or derivatives thereof, flavones, oxindoles, starurosporine, diarylureas, and paullones.

10. The method of claim 9, wherein the purine analog is roscovitine.

11. The method of claim 1, wherein the agent is and anti-Cdk2 antibody.

12. The method of claim 1, wherein the agent is a RNA mimetic.

13. The method of claim 1, wherein the agent is Nitric Oxide.

14. A method of reducing ischemia/reperfusion injury in a subject in need thereof comprising administering to the subject an agent that inhibits phosphorylation of retinoblastoma protein (Rb).

15. The method of claim 14, wherein the ischemia/reperfusion injury occurs following an ischemia/reperfusion event selected from the group consisting of myocardial inschemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypoxia, retinal ischemia, renal ischemia, ischemia/reperfusion events occurring during cardiac surgery where a heart lung machine is used such as Coronary artery bypassing, and ischemia/reperfusion events occurring during the preservation of organs for transplant.

16. The method of claim 14, wherein the agent is administered after the ischemia/reperfusion event.

17. The method of claim 16, wherein the agent is administered within 30 minutes, 1, 2, 6, 12, or 24 hour(s) following the ischemia/reperfusion event.

18. The method of claim 14, wherein the agent is administered before the ischemia/reperfusion event.

19. The method of claim 18, wherein the agent is administered at least 1, 2, 6, 12, or 24 hour(s) before the ischemia/reperfusion event.

20. The method of claim 18, wherein the agent is administered at least 2 days, 3 days, 1 week, or 2 weeks before the ischemia/reperfusion event.

21. The method of claim 14, wherein the agent is administered during the ischemia/reperfusion event.

22. The method of claim 14, wherein the agent is and anti-Rb antibody.

23. The method of claim 14, wherein the agent is a RNA mimetic.

24. A method of reducing infarct size following an ischemia/reperfusion event in a subject comprising administering to the subject an agent that inhibits Cdk2 activity.

25. The method of claim 24, wherein the ischemia/reperfusion event is selected from the group consisting of myocardial inschemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypoxia, retinal ischemia, renal ischemia, ischemia/reperfusion events occurring during cardiac surgery where a heart lung machine is used such as Coronary artery bypassing, and ischemia/reperfusion events occurring during the preservation of organs for transplant.

26. The method of claim 24, wherein the agent is administered after the ischemia/reperfusion event.

27. The method of claim 26, wherein the agent is administered within 30 minutes, 1, 2, 6, 12, or 24 hour(s) following the ischemia/reperfusion event.

28. The method of claim 24, wherein the agent is administered before the ischemia/reperfusion event.

29. The method of claim 28, wherein the agent is administered at least 1, 2, 6, 12, or 24 hour(s) before the ischemia/reperfusion event.

30. The method of claim 28, wherein the agent is administered at least 2 days, 3 days, 1 week, or 2 weeks before the ischemia/reperfusion event.

31. The method of claim 24, wherein the agent is administered during the ischemia/reperfusion event.

32. The method of claim 24, wherein the agent is selected from the group consisting of purine analogs or derivatives thereof, pyrimidine analogs or derivatives thereof, flavones, oxindoles, starurosporine, diarylureas, and paullones.

33. The method of claim 32, wherein the purine analog is roscovitine.

34. The method of claim 24, wherein the agent is and anti-Cdk2 antibody.

35. The method of claim 24, wherein the agent is a RNA mimetic.

36. The method of claim 24, wherein the agent is Nitric Oxide.

37. A method of reducing infarct size following an ischemia/reperfusion event in a subject comprising administering to the subject an agent that inhibits phosphorylation of Rb.

38. The method of claim 37, wherein the ischemia/reperfusion event is selected from the group consisting of myocardial inschemia, myocardial reperfusion, subendocardial ischemia, Takayasu's arteritis, stroke, ischemia strokes, cerebral thrombosis, cerebral embolism, atrial fibrillation, hemorrhagic strokes, aneurysm and arteriovenous malformation, transient ischemia attack, pulmonary infarction, hypoxia, retinal ischemia, renal ischemia, ischemia/reperfusion events occurring during cardiac surgery where a heart lung machine is used such as Coronary artery bypassing, and ischemia/reperfusion events occurring during the preservation of organs for transplant.

39. The method of claim 37, wherein the agent is administered after the ischemia/reperfusion event.

40. The method of claim 39, wherein the agent is administered within 30 minutes, 1, 2, 6, 12, or 24 hour(s) following the ischemia/reperfusion event.

41. The method of claim 37, wherein the agent is administered before the ischemia/reperfusion event.

42. The method of claim 41, wherein the agent is administered at least 1, 2, 6, 12, or 24 hour(s) before the ischemia/reperfusion event.

43. The method of claim 41, wherein the agent is administered at least 2 days, 3 days, 1 week, or 2 weeks before the ischemia/reperfusion event.

44. The method of claim 37, wherein the agent is administered during the ischemia/reperfusion event.

45. The method of claim 37, wherein the agent is and anti-Rb antibody.

46. The method of claim 37, wherein the agent is a RNA mimetic.

47. A method of screening for an agent that reduces infarct size following ischemia/reperfusion comprising administering an agent to a subject, inducing ischemia/reperfusion, and measuring the activity of Cdk2, wherein a decrease in the Cdk2 activity relative to a control indicates an agent that reduces infarct size.

48. The method of claim 47, wherein the ischemia/reperfusion is induced by ligation.

49. The method of claim 47, wherein the subject is a mammal.

50. The method of claim 49, wherein the mammal is selected from the group consisting of mouse, rat, rabbit, guinea pig, cow, horse, pig, cat, dog, monkey, chimpanzee, or human.

51. The method of claim 47, wherein Cdk2 activity is measured by western blot.

52. A method of screening for an agent that reduces ischemia/reperfusion injury comprising administering an agent to a subject, inducing ischemia/reperfusion, and measuring the activity of Cdk2, wherein a decrease in the Cdk2 activity relative to a control indicates an agent that reduces ischemia/reperfusion injury.

53. The method of claim 52, wherein the ischemia/reperfusion is induced by ligation.

54. The method of claim 52, wherein the subject is a mammal.

55. The method of claim 54, wherein the mammal is selected from the group consisting of mouse, rat, rabbit, guinea pig, cow, horse, pig, cat, dog, monkey, chimpanzee, or human.

56. The method of claim 52, wherein Cdk2 activity is measured by western blot.


SEQUENCE LISTING (6 sequences)

SEQ ID NO: 1 - DNA, 7642 bp, Artificial Sequence
  Description of Artificial Sequence/note = synthetic construct
agacagaaacagaaccctgcatcccaaaacagagacagtgtcggggtatgctatgaggcagaggcaacgactcctctgaggcccaggattcctttgcaacgagattcccggcttcctggtttccaaaggaggctcctgggccaggtggaatttaaccttaggtcgatacatattttccccaggattggtgaaaatagtgagttagaaatcaagaaatccctacagactagagacataggtaggaaacttggacccaaagcaggtacttgggaagagtgttcagccgccccaccggggaagaaacttgtctagcccccaagaaccaaacttgcctggggcgggatcatttgcggggaggagcgctgggctcgccccccggctctgacgttgaccaatagaaaggcctgggggcggagccagggaaacgcgggaagcaggggcggggcctctggtggcggtcgggaactcggtgggaggcggcaacattgtttcaagttggccaaattgacaagagcgagaggtatactgcgttccatcccgacccggggccacggtactgggccctgtttccccctcctcggcccccgagagccagggtccgccttctgcagggttcccaggcccccgctccagggccgggctgacccgactcgctggcgcttcatggagaacttccaaaaggtggaaaagatcggagagggcacgtacggagttgtgtacaaagccagaaacaagttgacgggagaggtggtggcgcttaagaaaatccgcctggacacgtgagtggcctctgtacccgggactcctaactggggacctccttgattgtcccccccaaccccccacgggcgggtagccgtccagggaccggaagagagcagggagggacttctttagaagtggagaggtgggttgggggccagtagaaggtgaagagtatacttatactccctggggagagtatagggtggtgtggaatccatggaaaactttcttcccaaactgagccggatcgtgcccccaaatgtgcgactacagactcggggagagaaaggaggtctctgagatgaggtccaagactctccatggagtggagttatgtgggaaccggcgagaatcgcctttctgaatgaagagccctcttcactgccccaccctcaccttagaattctctcctctttccaaagaatggcagttgaacctcactggcccctctggggaggctgggggctactcctgcattttttcccctccattacagtctccctgcttcaccttcaccaggcggctttacttacctacccctgggaaaagaggagataatggccttaatatatccaaaaaccacaccctgactacccaagaattagctcttaccatcaccctttctcttctctcactttcctagggggtgctgggtggtgtctccttgggggaaagaaatgactaggtgggggggaaaggaatatttgtaaccatattcccatctctgctttcccaacctctccaagtgagactgagggtgtgcccagtactgccatccgagagatctctctgcttaaggagcttaaccatcctaatattgtcaagtaagtatgcgtctgagaggtgatccagctggaaaggaggataagttctgtctgtacagtgtgggcatttctctctctcacacacctccatttcctcaaactttccttctctaggctgctggatgtcattcacacagaaaataaactctacctggtttttgaatttctgcaccaagatctcaagaaattcatggatgcctctgctctcactggcattcctcttcccctcatcaaggtaatgcttctcatcagctcctctcatcatgggcatgtcttgggggactggtggcaggcaattcagggtgatattttatgattttggcctccttctgagccctcatctcctatacacacacactccccttctttttgtgtctccttccctgctcattatattcattaaccctagggttggactgaacaatcaaagttgaaactctagtgagtcaacctagcaactcaggtgggaggtcagatgaaactcagataaacgggatttgagagcacttggtaaattcctccaaaaagcccttccatttggtggaagacctagctagtgagtccctattgtctattttagggctggattcttcactcccagagctactttcaatctattaacaaacattttttcaatgcacaggatgtagaaaagggatggaaaattgagtaagacttggtccttatcctctctgggctgacagtccattgggagaaatagcttgtaaatatgtaactataatccaacataataaaggctttagtagagttttaggggcacagagcaaacccagtctgctcactgtaatggagaaacacagtcctctctttctcctttgtcagagctatctgttccagctgctccagggcctagctttctgccattctcatcgggtcctccaccgagaccttaaacctcagaatctgcttattaacacagagggggccatcaagctagcagactttggactagccagagcttttggagtccctgttcgtacttacacccatgaggtgagtccctttatgtcttttttctctgagcttcccaagaggtgttaactagggtattcacaaagttactaaaaatatctggctaacagtttcttactaggtagaaataatctcttgacatcctaaagagtcttagggtatgcatggaattcatactgtgttgctaactgggcccacacctgtaataccaatactttgggaggctgaggtgggaggatcacttgagcccaggagttcgagaccatcatgggcaacatagcgagaccccatctctacaaatctacaaaaagaaaaaatttagaaataaaattatgaccaatttgtctcaagtttttccaggaagatctcaaattaggggttcagtccagaactatggactggaaatcagtgggaggggaaagatgatggagggaaggaaactgcttgttaagaggccaagagtaagcagagtagtgttgaggaactgagatgcgggaatttccataccctataaaccaccccgcccctccctattcccgtccctcaggtggtgaccctgtggtaccgagctcctgaaatcctcctgggctgcaaatattattccacagctgtggacatctggagcctgggctgcatctttgctgagatggtatggaggcttgcccaagttccacccagccccctccctctcctccccacatccaagaacaacagaactgcttcttggcccagacctatggcccttctatcacagggttctctctctaaagtagcaccaaggggaatggtgggaaaggatgcaactgttgccctgatatcaaccacagtgttaggatatcctcaaacagccttagtacctggtatacatctcttatccctgaaataagttaaagcatttctgcagctgttttagctgtagtctgcatatatttgggagaatgattccatttagtgcctcttttatttcaggccttcatttcaaggcttgtagaccttgttgtatggtgccagcaatgtagtgaagacaactgtggtcactttacccacacctttcatttaaactgcagatttaggcagggtgcagtggctcacacctataataccagcactttgggaggctgaggtaggtggatcacctgaggtcaggagtttgagaccagcctggccaacatgttaaaaccctgtctctactaaaaatacaaaaattagccaggtgtggctacttgggattacacacctgtaatcccagctacttgggaggccaaggcaggagaatcggttgaacccgggagatggaggttgcagtgaccaagattgcaccactgcactccagcctgggcgacagaatgagattccatctcaaaaaaaaaaaaaaaaaaaaaaaaaaaaagatttagatcatgttccccttcaacctctggcttttcagactgaaggatccttgaagcctggctttatgtagaagctcccatctcctttaatataacagtacagtggtgcagtaggctgtcttcaaatcagcaatatgttttattgtcttttatcttggttgtaaccaagagcttaaagaccattagcctatacatatgtaatgtgcatttatccccccagtgcattaccttacaattgtccgtattcctctctcaattcatcaaaaaatatttgttaagcacctagtgggtacccagcaccatgctaggtgctgtggggaacacagaagaaatggaagacagagtctctgcccgctgtgctcgtatctagaagtggctgcatcacaaggttgggggatgaccgcagtgtctaccccctaccccgtgagtggcttgggatacctttgctacatgtcagtggcaccccagacattcaccccctcccagacccacccagccttggggatctgcaaagccatggttgggggaaggaaggagggggcgaggagacagatgaaggaacttcattgtctcaggttctgtgtgactgaccccatgaaaggccctggggagggagtcatggggccctgctgaccttttactgtctgtgggaactcctttgtatagaggagagttttgactgacgtcaacgtgggtcttggtatttcctctttccccattttcaggtgactcgccgggccctattccctggagattctgagattgaccagctcttccggatctttcggactctggggaccccagatgaggtggtgtggccaggagttacttctatgcctgattacaagccaagtttccccaagtgggcccggcaagattttagtaaagttgtacctcccctggatgaagatggacggagcttgttatcggtgagagtgggcacctgttttccctcattcatttctcccagggaagggcttttccaggatgaaggaaggatgagaccctgaaatctgggcctcagtgtttcatttccctggttcctgctctccctgttggcacactgattcagctatgggaggatggaagtgagaattctgccttgggtagaaggagttctggtttcctgatttctgggaacacctgctgcccatttagtccactatcacatcattgaagtcaacatgcatctctccctctagcaaatgctgcactacgaccctaacaagcggatttcggccaaggcagccctggctcaccctttcttccaggatgtgaccaagccagtaccccatcttcgactctgatagccttcttgaagcccccagccctaatctcaccctctcctccagtgtgggcttgaccaggcttggccttgggctatttggactcaggtgggccctctgaacttgccttaaacactcaccttctagtcttggccagccaactctgggaatacaggggtgaaaggggggaaccagtgaaaatgaaaggaagtttcagtattagatgcacttaagttagcctccaccaccctttcccccttctcttagttattgctgaagagggttggtataaaaataattttaaaaaagccttcctacacgttagatttgccgtaccaatctctgaatgccccataattattatttccagtgtttgggatgaccaggatcccaagcctcctgctgccacaatgtttataaaggccaaatgatagcgggggctaagttggtgcttttgagaaccaagtaaaacaaaaccactgggaggagtctattttaaagaattcggttgaaaaaatagatccaatcagtttataccctagttagtgttttgcctcacctaataggctgggagactgaagactcagcccgggtggggctgcagaaaaatgattggccccagtccccttgtttgtcccttctacaggcatgaggaatctgggaggccctgagacagggattgtgcttcattccaatctattgcttcaccatggccttatgaggcaggtgagagatgtttgaatttttctcttccttttagtattcttagttgttcagttgccaaggatccctgatcccattttcctctgacgtccacctcctaccccataggagttagaagttagggtttaggcatcattttgagaatgctgacactttttcagggctgtgattgagtgagggcatgggtaaaaatatttctttaaaagaaggatgaacaattatatttatatttcaggttatatccaatagtagagttggctttttttttttttttttggtcatagtgggtggatttgttgccatgtgcaccttggggttttgtaatgacagtgctaaaaaaaaaaagcattttttttttatgatttgtctctgtcacccttgtccttgagtgctcttgctattaacgttatttgtaatttagtttgtagctcattaaaaaaatgtgcctagttttatagttcatctctttcctcctcttatttactgaaagaatggtgggagacagaatatgaatgtgtgtatgtgatcattgatttcagaccccttttcctaatctctctgtgcatggctgcccaccccaccccattttggctagtgctgacggtgaacaaagtggttattatcatccctatttatggctggggaaactgaagcctatatacttcctacttcactttctccacacaccagcaaacctcctttcccccagccaacataccacacacacaccacaccacacacacatcacatacacacaccacacacacatcatatacacacatataccacacacaccacgtatacatataccaaatacacacacaccacatacacaccatatatgcacacacaccccatacatacacacacccatgcacatacatagcggcaggaagagggctcccggggaatttgcctttcgacactgcatttcccagtaagcccgccgtttttcctttctgtttcatatcacagacagaaaagttttccaaagattcttttatttacatttacatacgattctgaaaaagcgtctccaagctaccacttcttgtagttgccattgtctcattccaccaaaaaacatctcagaccgcccgcggccaccccaaatacgcctgtccactgaagcctcactaaaaggaggaagcgctgatgtcagggttacttaaattcccaggctccggatccctagaagaggtatttccctttacgcccgcgaatttgtcatagaaggcagggcactagagccagccccagccaagtccagggggaggggcgaaggtcagtagcggccttcctaaggcttccttcaacccctccttgcctgccagtcaagtaccgtattttcgcagtactggttaaagtttcactttgacccgcctccatcactcacctgattagatgtttatgagtcaaaaggcggggtcatggggtgtcgagattttattggtggatacatacgcctgtcggcagtgtaccgccccctgtcttcaggagcttgctctttaatggctccttacctcgtccatcttcgaggtttctccctcgctattggcttgtggccccgcctgtcgcgtcccttgctcgtacttgttggtgacagcgcccaaagagactcgcctttctccagaggattggtggatgagtgcacatatag

SEQ ID NO: 2 - protein, 298 aa, Artificial Sequence
  Description of Artificial Sequence/note = synthetic construct
MENFQKVEKIGEGTYGVVYKARNKLTGEVVALKKIRLDTETEGVPSTAIREISLLKELNHPNIVKLLDVIHTENKLYLVFEFLHQDLKKFMDASALTGIPLPLIKSYLFQLLQGLAFCHSHRVLHRDLKPQNLLINTEGAIKLADFGLARAFGVPVRTYTHEVVTLWYRAPEILLGCKYYSTAVDIWSLGCIFAEMVTRRALFPGDSEIDQLFRIFRTLGTPDEVVWPGVTSMPDYKPSFPKWARQDFSKVVPPLDEDGRSLLSQMLHYDPNKRISAKAALAHPFFQDVTKPVPHLRL

SEQ ID NO: 3 - DNA, 2994 bp, Artificial Sequence
  Description of Artificial Sequence/note = synthetic construct
ttccggtttttctcaggggacgttgaaattatttttgtaacgggagtcgggagaggacggggcgtgccccgcgtgcgcgcgcgtcgtcctccccggcgctcctccacagctcgctggctcccgccgcggaaaggcgtcatgccgcccaaaaccccccgaaaaacggccgccaccgccgccgctgccgccgcggaacccccggcaccgccgccgccgccccctcctgaggaggacccagagcaggacagcggcccggaggacctgcctctcgtcaggcttgagtttgaagaaacagaagaacctgattttactgcattatgtcagaaattaaagataccagatcatgtcagagagagagcttggttaacttgggagaaagtttcatctgtggatggagtattgggaggttatattcaaaagaaaaaggaactgtggggaatctgtatctttattgcagcagttgacctagatgagatgtcgttcacttttactgagctacagaaaaacatagaaatcagtgtccataaattctttaacttactaaaagaaattgataccagtaccaaagttgataatgctatgtcaagactgttgaagaagtatgatgtattgtttgcactcttcagcaaattggaaaggacatgtgaacttatatatttgacacaacccagcagttcgatatctactgaaataaattctgcattggtgctaaaagtttcttggatcacatttttattagctaaaggggaagtattacaaatggaagatgatctggtgatttcatttcagttaatgctatgtgtccttgactattttattaaactctcacctcccatgttgctcaaagaaccatataaaacagctgttatacccattaatggttcacctcgaacacccaggcgaggtcagaacaggagtgcacggatagcaaaacaactagaaaatgatacaagaattattgaagttctctgtaaagaacatgaatgtaatatagatgaggtgaaaaatgtttatttcaaaaattttataccttttatgaattctcttggacttgtaacatctaatggacttccagaggttgaaaatctttctaaacgatacgaagaaatttatcttaaaaataaagatctagatgcaagattatttttggatcatgataaaactcttcagactgattctatagacagttttgaaacacagagaacaccacgaaaaagtaaccttgatgaagaggtgaatgtaattcctccacacactccagttaggactgttatgaacactatccaacaattaatgatgattttaaattcagcaagtgatcaaccttcagaaaatctgatttcctattttaacaactgcacagtgaatccaaaagaaagtatactgaaaagagtgaaggatataggatacatctttaaagagaaatttgctaaagctgtgggacagggttgtgtcgaaattggatcacagcgatacaaacttggagttcgcttgtattaccgagtaatggaatccatgcttaaatcagaagaagaacgattatccattcaaaattttagcaaacttctgaatgacaacatttttcatatgtctttattggcgtgcgctcttgaggttgtaatggccacatatagcagaagtacatctcagaatcttgattctggaacagatttgtctttcccatggattctgaatgtgcttaatttaaaagcctttgatttttacaaagtgatcgaaagttttatcaaagcagaaggcaacttgacaagagaaatgataaaacatttagaacgatgtgaacatcgaatcatggaatcccttgcatggctctcagattcacctttatttgatcttattaaacaatcaaaggaccgagaaggaccaactgatcaccttgaatctgcttgtcctcttaatcttcctctccagaataatcacactgcagcagatatgtatctttctcctgtaagatctccaaagaaaaaaggttcaactacgcgtgtaaattctactgcaaatgcagagacacaagcaacctcagccttccagacccagaagccattgaaatctacctctctttcactgttttataaaaaagtgtatcggctagcctatctccggctaaatacactttgtgaacgccttctgtctgagcacccagaattagaacatatcatctggacccttttccagcacaccctgcagaatgagtatgaactcatgagagacaggcatttggaccaaattatgatgtgttccatgtatggcatatgcaaagtgaagaatatagaccttaaattcaaaatcattgtaacagcatacaaggatcttcctcatgctgttcaggagacattcaaacgtgttttgatcaaagaagaggagtatgattctattatagtattctataactcggtcttcatgcagagactgaaaacaaatattttgcagtatgcttccaccaggccccctaccttgtcaccaatacctcacattcctcgaagcccttacaagtttcctagttcacccttacggattcctggagggaacatctatatttcacccctgaagagtccatataaaatttcagaaggtctgccaacaccaacaaaaatgactccaagatcaagaatcttagtatcaattggtgaatcattcgggacttctgagaagttccagaaaataaatcagatggtatgtaacagcgaccgtgtgctcaaaagaagtgctgaaggaagcaaccctcctaaaccactgaaaaaactacgctttgatattgaaggatcagatgaagcagatggaagtaaacatctcccaggagagtccaaatttcagcagaaactggcagaaatgacttctactcgaacacgaatgcaaaagcagaaaatgaatgatagcatggatacctcaaacaaggaagagaaatgaggatctcaggaccttggtggacactgtgtacacctctggattcattgtctctcacagatgtgactgtat

SEQ ID NO: 4 - protein, 928 aa, Artificial Sequence
  Description of Artificial Sequence/note = synthetic construct
MPPKTPRKTAATAAAAAAEPPAPPPPPPPEEDPEQDSGPEDLPLVRLEFEETEEPDFTALCQKLKIPDHVRERAWLTWEKVSSVDGVLGGYIQKKKELWGICIFIAAVDLDEMSFTFTELQKNIEISVHKFFNLLKEIDTSTKVDNAMSRLLKKYDVLFALFSKLERTCELIYLTQPSSSISTEINSALVLKVSWITFLLAKGEVLQMEDDLVISFQLMLCVLDYFIKLSPPMLLKEPYKTAVIPINGSPRTPRRGQNRSARIAKQLENDTRIIEVLCKEHECNIDEVKNVYFKNFIPFMNSLGLVTSNGLPEVENLSKRYEEIYLKNKDLDARLFLDHDKTLQTDSIDSFETQRTPRKSNLDEEVNVIPPHTPVRTVMNTIQQLMMILNSASDQPSENLISYFNNCTVNPKESILKRVKDIGYIFKEKFAKAVGQGCVEIGSQRYKLGVRLYYRVMESMLKSEEERLSIQNFSKLLNDNIFHMSLLACALEVVMATYSRSTSQNLDSGTDLSFPWILNVLNLKAFDFYKVIESFIKAEGNLTREMIKHLERCEHRIMESLAWLSDSPLFDLIKQSKDREGPTDHLESACPLNLPLQNNHTAADMYLSPVRSPKKKGSTTRVNSTANAETQATSAFQTQKPLKSTSLSLFYKKVYRLAYLRLNTLCERLLSEHPELEHIIWTLFQHTLQNEYELMRDRHLDQIMMCSMYGICKVKNIDLKFKIIVTAYKDLPHAVQETFKRVLIKEEEYDSIIVFYNSVFMQRLKTNILQYASTRPPTLSPIPHIPRSPYKFPSSPLRIPGGNIYISPLKSPYKISEGLPTPTKMTPRSRILVSIGESFGTSEKFQKINQMVCNSDRVLKRSAEGSNPPKPLKKLRFDIEGSDEADGSKHLPGESKFQQKLAEMTSTRTRMQKQKMNDSMDTSNKEEK

SEQ ID NO: 5 - protein, 14 aa, Artificial Sequence
  Description of Artificial Sequence/note = synthetic construct
DIEGADEADGSKHL

SEQ ID NO: 6 - protein, 14 aa, Artificial Sequence
  Description of Artificial Sequence/note = synthetic construct
DIEGADEAAESKHL